(19) United States
(12) Patent Application Publication
(10) Pub. No.: US 2013/0072751 A1
(43) Pub. Date: Mar. 21, 2013

(54) Inflatable Penile Prosthesis with Unbiased Check Valve

(71) Applicant: Terence M. Fogarty, Hudson, WI (US)

(72) Inventor: Terence M. Fogarty, Hudson, WI (US)

(21) Appl. No.: 13/467,024

(22) Filed: May 8, 2012

Related U.S. Application Data
(60) Provisional application No. 61/535,781, filed on Sep. 16, 2011.

(51) Int. Cl. A61F 2/26; F16K 21/04

(52) U.S. Cl. 600/40; 137/511

(57) ABSTRACT

According to various embodiments, an IPP includes unbiased one-way check valve assemblies either independently or in conjunction with other valve assemblies to direct fluid into and evacuate fluid out of inflatable penile cylinders. The fluid transfer system transfers fluid from the fluid reservoir to inflatable penile cylinders and includes a fluid transfer bulb, unbiased inlet valve assembly, unbiased exhaust valve assembly and spool valve assembly. The cylinder deflate mechanism evacuates fluid from the penile cylinders to the reservoir and includes a deflate actuator, unbiased return valve assembly, and spool valve assembly.

### TECHNICAL FIELD

This disclosure relates generally to prosthetic medical devices and methods of using such devices in implant surgery. More particularly, this disclosure relates to implantable inflatable penile prostheses used to treat male impotence.

### BACKGROUND

Historically, implantable penile prostheses, both the inflatable type and non-inflatable type that include rigid, mechanical and malleable devices have been supplied with paired penile cylinders. Inflatable penile prosthesis (IPP) devices typically include a pair of inflatable penile cylinders, a fluid transfer pump, a deflate mechanism, a fluid storage reservoir and flexible tubing connecting components. There are three basic categories of IPP devices: one-piece, two-piece, and three-piece IPP devices. One-piece IPP devices are configured with an inflatable bladder, fluid transfer pump, deflate mechanism and fluid reservoir wholly contained in each of the two unitary paired penile cylinders that are implanted in their entirety in the corpora cavernosa of the penis. Two-piece IPP devices are configured with paired inflatable penile cylinders implanted in the corpora cavernosa of the penis. At least one of the other components (e.g., the fluid transfer pump, the deflate mechanism, or the fluid reservoir) is implanted outside the corpora cavernosa. Three-piece IPP devices are configured with paired inflatable penile cylinders implanted in the corpora cavernosa of the penis. At least two of the other components (e.g., the fluid transfer pump, the deflate mechanism, or the fluid reservoir) are implanted outside the corpora cavernosa.

One-piece and two-piece IPP devices have included a fluid storage chamber or reservoir within the cylinder component implanted in the corpus cavernosum, with the reservoir configured distal to the inflatable bladder as disclosed by Burton et al. in U.S. Pat. No. 4,267,829. The reservoir has been configured proximal to the inflatable bladder (e.g., Finney, U.S. Pat. No. 4,318,396), within inflatable bladders (e.g., Whitehead, U.S. Pat. No. 4,665,903), or surrounding the inflatable bladder (e.g., Finney, U.S. Pat. No. 4,353,360). Two-piece IPP devices have also been configured with a reservoir implanted outside the corpora cavernosa and supplying fluid to one or both inflatable penile cylinders (e.g., Uson, U.S. Pat. No. 4,009,711). Three-piece IPP devices have been configured with a single reservoir implanted outside the corpora cavernosa and supplying fluid to both penile inflatable cylinders (e.g., Buuck, U.S. Pat. No. 3,954,102). Each of the cited patents is incorporated by reference in its entirety.

### SUMMARY

The present disclosure relates to a departure from the design of check valve assemblies, typically one-way check valves, used with fluid transfer systems and cylinder deflate mechanisms previously used in one, two and three-piece IPP devices. An IPP device is disclosed comprising at least one unbiased one-way check valve assembly having a valve component with a specific gravity substantially similar to the specific gravity of the fluid used in said IPP device. According to various embodiments disclosed herein, unbiased check valve assemblies are used either independently or in conjunction with other type valve assemblies to direct fluid into and evacuate fluid out of inflatable penile cylinders. The fluid transfer system transfers fluid from the fluid reservoir to inflatable penile cylinders and may include a fluid transfer bulb, an unbiased inlet valve assembly and an unbiased exhaust valve assembly. The cylinder deflate mechanism evacuates fluid from the penile cylinders to the fluid reservoir and may include unbiased check valve assemblies. Unbiased check valve assemblies may be used to prevent flow from the fluid reservoir to the cylinders or from the cylinders to the fluid reservoir.

The principles disclosed herein can be practiced in connection with, for example, a one-piece IPP device implanted in the corpus cavernosum, or two-piece or three-piece IPP devices having paired inflatable penile cylinders implanted in the corpora cavernosa of the penis.

In a two-piece IPP configuration, two inflatable penile cylinders are implanted in the corpora cavernosa of the penis. A multifunctional fluid reservoir, including a fluid reservoir, fluid transfer system and cylinder deflate mechanism are implanted subcutaneously in the lower abdomen. Flexible tubing provides fluid communication between the inflatable penile cylinders, the fluid transfer system and the cylinder deflate mechanism incorporated in the multifunctional fluid reservoir. Using flexible tubing, the fluid transfer bulb and/or the deflate actuator bulb may be located remote from other components of the fluid transfer system and deflate mechanism respectively that are contained within the multifunctional reservoir. With either the fluid transfer bulb or deflate actuator bulb located remote from the multifunctional reservoir, the device might not be classified as a two-piece device.

In a three-piece IPP configuration, two inflatable penile cylinders are implanted in the corpora cavernosa of the penis. The fluid transfer system and deflate mechanism are combined into a unitary fluid transfer pump that is implanted in the scrotum. The fluid transfer system includes an unbiased inlet valve assembly, an unbiased exhaust valve assembly, and a spool valve assembly. The cylinder deflate mechanism has an integral deflate actuator button, an unbiased return valve assembly and a spool valve assembly. A single spool valve assembly functions for both the fluid transfer system and deflate mechanism. A fluid reservoir is implanted in the abdomen, either in the Space of Retzius or subcutaneously in the lower abdomen. Flexible tubing provides fluid communication between the inflatable penile cylinders and the fluid reservoir placed in the abdomen and the fluid transfer pump placed in the scrotum.

Two-piece or three-piece IPP devices described above may be provided as components that require assembly using tubing connectors by the surgeon at implantation or as fully assembled connector-less prostheses. Assembled devices may be provided unfilled or prefilled with physiological-type solution such as saline or radiopaque solutions. Devices may be provided either sterile or non-sterile. Sterile devices may be provided in packages having multiple barriers for presentation in a sterile field.

One embodiment is directed to an IPP comprising a fluid reservoir, at least one inflatable penile cylinder, and a fluid transfer system in fluid communication with the fluid reservoir and the at least one inflatable penile cylinder to inflate the at least one penile cylinder with fluid from the fluid reservoir. The fluid transfer system includes an unbiased inlet valve assembly in fluid communication with the fluid reservoir, a fluid transfer bulb in fluid communication with the unbiased inlet valve assembly, an unbiased exhaust valve assembly in fluid communication with the fluid transfer bulb and at least one inflatable penile cylinder, and a spool valve assembly in fluid communication with the fluid transfer bulb and at least one inflatable penile cylinder. The IPP also includes a cylinder deflate mechanism in fluid communication with the fluid reservoir and with the at least one inflatable penile cylinder to return fluid from the at least one inflatable penile cylinder to the fluid reservoir. The cylinder deflate mechanism includes the spool valve assembly, an unbiased return valve assembly in fluid communication with the reservoir and the at least one inflatable penile cylinder, and a deflate actuator arranged to shift the spool valve assembly to a cylinder deflate mode. The spool valve assembly can be shifted between a cylinder inflate mode and the cylinder deflate mode using a force external to the spool valve assembly.

Another embodiment is directed to a two-piece IPP comprising a multifunctional fluid reservoir comprising a fluid transfer system to inflate at least one penile cylinder and a cylinder deflate mechanism to return fluid from the at least one inflatable penile cylinder to the fluid reservoir. The fluid transfer system comprises an unbiased inlet valve assembly in fluid communication with a fluid reservoir, a fluid transfer bulb in fluid communication with the unbiased inlet valve assembly, an unbiased exhaust valve assembly in fluid communication with the fluid transfer bulb and the at least one inflatable penile cylinder, and a spool valve assembly in fluid communication with the fluid transfer bulb and the at least one inflatable penile cylinder. The cylinder deflate mechanism comprises the spool valve assembly, an unbiased return valve assembly in fluid communication with the reservoir and the at least one inflatable penile cylinder, and a deflate actuator to shift the spool valve assembly to a cylinder deflate mode. At least one inflatable penile cylinder is in fluid communication with the multifunctional fluid reservoir. The spool valve assembly can be shifted between a cylinder inflate mode and the cylinder deflate mode using external force applied to a spool within the spool valve assembly.

Still another embodiment is directed to a three-piece (IPP) comprising a fluid reservoir, at least one inflatable penile cylinder in fluid communication with the fluid reservoir, and a fluid transfer pump in fluid communication with the fluid reservoir and the at least one inflatable penile cylinder. The fluid transfer pump comprising a fluid transfer system and a deflate mechanism. The fluid transfer system comprises an unbiased inlet valve assembly in fluid communication with the fluid reservoir, a fluid transfer bulb in fluid communication with the unbiased inlet valve assembly, an unbiased exhaust valve assembly in fluid communication with the fluid transfer bulb and the at least one inflatable penile cylinder, and a spool valve assembly in fluid communication with the fluid transfer bulb and the at least one inflatable penile cylinder. The deflate mechanism comprises a spool valve assembly in fluid communication with the fluid reservoir and the at least one inflatable penile cylinder, an unbiased return valve assembly in fluid communication with the reservoir and the at least one inflatable penile cylinder, and a deflate actuator button to mechanically shift the spool valve assembly to a cylinder deflate mode. The spool valve assembly can be shifted between a cylinder inflate mode and the cylinder deflate mode using a force external to the spool valve assembly.

Yet another embodiment is directed to an IPP comprising a fluid reservoir, at least one inflatable penile cylinder, a fluid transfer system to inflate the at least one penile cylinder with fluid from the fluid reservoir, and a deflate mechanism to return fluid from the at least one inflatable penile cylinder to the fluid reservoir. The fluid transfer system is positioned to provide fluid communication between the fluid reservoir and the at least one inflatable penile cylinder. The deflate mechanism is positioned to provide fluid communication between the at least one inflatable penile cylinder and the fluid reservoir. The fluid transfer system and the deflate mechanism include a shared spool valve assembly.

Another embodiment is directed to an IPP comprising a fluid reservoir, at least one inflatable penile cylinder, and a fluid transfer system including a fluid transfer pump, a deflate mechanism and a shared spool valve assembly. The shared spool valve assembly comprises a housing with cylindrical bore and fluid ports associated with the fluid transfer system and deflate mechanism and a spool capable of shifting axially within the housing to cooperate with the fluid ports in the housing. The spool includes a fluid transfer system channel, an unbiased exhaust valve assembly located within the fluid transfer channel in the spool to prevent backflow from the at least one inflatable penile cylinder to the fluid transfer pump, a deflate mechanism fluid channel, and an unbiased return valve assembly located within the deflate mechanism fluid channel in the spool to prevent backflow from the fluid reservoir to the at least one inflatable penile cylinder. The spool valve assembly can be shifted between a cylinder inflate mode and a cylinder deflate mode using a force external to the spool valve assembly.

Yet another embodiment is directed to an IPP comprising a fluid reservoir having a substantially flat posterior base comprising a reinforced elastomeric base to maintain dimensional stability and at least one inflatable penile cylinder. A fluid transfer system inflates the at least one inflatable penile cylinder with fluid from the fluid reservoir. A deflate mechanism returns fluid from the at least one inflatable penile cylinder to the fluid reservoir.

While multiple embodiments are disclosed, still other embodiments will become apparent to those skilled in the art from the following detailed description, which shows and describes illustrative embodiments. Accordingly, the drawings and detailed description are to be regarded as illustrative in nature and not restrictive.

### BRIEF DESCRIPTION OF THE DRAWINGS

FIG. 1 is a schematic of an example IPP device incorporating a spool valve assembly and three unbiased check valve assemblies depicted in a cylinder inflate mode according to one embodiment.

FIG. 2 is a schematic of the IPP device of FIG. 1 with the spool valve assembly and three unbiased check valve assemblies depicted in the cylinder deflate mode.

FIG. 3 is a schematic of an example IPP device incorporating a spool valve assembly and three unbiased check valve assemblies depicted in the cylinder inflate mode according to another embodiment.

FIG. 4 is a schematic of the IPP device of FIG. 3 with the spool valve assembly and three unbiased check valve assemblies depicted in the cylinder deflate mode.

FIG. 5 depicts an implanted two-piece IPP device in a flaccid state.

FIG. 6 depicts a two-piece IPP device with multifunctional reservoir with the penile cylinders in the flaccid state.

FIG. 7 depicts the two-piece IPP device of FIG. 6 with the penile cylinders in the erectile state.

FIG. 8 depicts the multifunctional reservoir of FIG. 6 with an unbiased inlet valve assembly, spool valve assembly, fluid transfer bulb, deflate actuator bulb, fluid fill port, and flexible tubing connecting the components exposed.

FIG. 9 is a sectional view of the spool valve assembly with two unbiased check valve assemblies used with a deflate actuator bulb depicted in FIG. 8, with the spool valve assembly in the cylinder deflate mode.

FIG. 10 is a sectional view of the spool valve assembly with two unbiased check valve assemblies used with a deflate actuator bulb depicted in FIG. 8, with the spool valve assembly in the cylinder inflate mode.

FIG. 11 depicts a multifunctional reservoir according to another embodiment, with the spool valve assembly, fluid transfer bulb, deflate actuator button, fluid fill port and flexible tubing connecting the components exposed.

FIG. 12 is a sectional view of the spool valve assembly with two unbiased check valve assemblies used with a deflate actuator button depicted in FIG. 11, with the spool valve assembly in the cylinder deflate mode.

FIG. 13 is a sectional view of the spool valve assembly with two unbiased check valve assemblies used with a deflate actuator button depicted in FIG. 11, with the spool valve assembly in the cylinder inflate mode.

FIG. 14 depicts an example implanted three-piece IPP device in the flaccid state according to one embodiment.

FIG. 15 is a cross-sectional view of the fluid transfer pump with integral cylinder deflate mechanism having a spool valve assembly with two unbiased check valve assemblies in the cylinder deflate mode as depicted in FIG. 14.

FIG. 16 depicts the implanted three-piece IPP device of FIG. 14 in the erectile state.

FIG. 17 is a sectional view of the fluid transfer pump with integral cylinder deflate mechanism having a spool valve assembly with two unbiased check valve assemblies in the cylinder inflate mode as depicted in FIG. 16.

FIG. 18 depicts an example implanted three-piece IPP in the flaccid state.

FIG. 19 is a cross-sectional view of the fluid transfer pump with integral cylinder deflate mechanism having a spool valve assembly with two unbiased check valve assemblies in the cylinder deflate mode as depicted in FIG. 18.

FIG. 20 depicts the implanted three-piece IPP of FIG. 18 in the erectile state.

FIG. 21 is a cross-sectional view of the fluid transfer pump with integral cylinder deflate mechanism having a spool valve assembly with two unbiased check valve assemblies in the cylinder inflate mode as depicted in FIG. 20.

FIG. 22 depicts an example IPP with a substantially round multifunctional reservoir, remote fluid transfer bulb and penile cylinders in the flaccid state according to another embodiment.

FIG. 23 depicts an example IPP with a substantially trapezoidal multifunctional reservoir, remote fluid transfer bulb and penile cylinders in the flaccid state according to still another embodiment.

FIG. 24 depicts the IPP of FIG. 22 in the flaccid state with portions of the anterior wall of the reservoir removed to expose components within the multi-functional reservoir.

FIG. 25 depicts the IPP of FIG. 23 in the flaccid state with portions of the anterior wall of the reservoir removed to expose components within the multi-functional reservoir.

FIG. 26 is a sectional view through the axis of an unbiased check valve assembly with both the entry and exit ports penetrating opposite ends of the valve chamber.

FIG. 26a is a sectional view perpendicular to the axis of an unbiased check valve depicted in FIG. 26, depicting the exit port and chamber end of the valve chamber.

FIG. 27 is section view of an unbiased check valve assembly with the entry port penetrating a valve chamber end and the exit port penetrating the chamber wall.

### DETAILED DESCRIPTION

An implantable IPP can be a unitary or multi-component device that is surgically implanted in a male patient to artificially achieve an erection of the penis for treatment of erectile dysfunction. IPP devices operate hydraulically and may include at least one inflatable penile cylinder, a fluid reservoir that is in fluid communication with the at least one inflatable penile cylinder, a fluid transfer system to transfer fluid from the reservoir to the at least one inflatable penile cylinder, and a deflate mechanism for returning fluid from the at least one inflatable penile cylinder to the fluid reservoir.

The IPP device can be operated in an inflate mode and in a deflate mode. In the inflate mode, also known as a cylinder inflate mode or device inflate mode, the cylinder or device inflates. The inflate mode is initiated by a volitional deformation of the fluid transfer bulb that closes the unbiased inlet valve assembly and opens the unbiased exhaust valve assembly. Fluid from the fluid transfer bulb shifts the spool within the spool valve assembly to the inflate position. Once the spool is shifted to the inflate position, it remains in the inflate mode during recovery and repetitive deformation of the fluid transfer bulb, while the inflatable penile cylinders are filled and pressurized to the desired erectile state. During pump bulb recovery, the unbiased inlet valve assembly opens and the unbiased exhaust valve assembly closes. Subsequent to operating the fluid transfer bulb, the spool valve assembly remains in the inflate mode until the spool is volitionally shifted to the deflate mode.

In the deflate mode, also known as a cylinder deflate mode or device deflate mode, the cylinder or device deflates. The deflate mode is initiated by the volitional actuation of either a deflate actuator button or a deflate actuator bulb. The deflate actuator applies force, either mechanical with the button or pilot pressure with the bulb, to shift the spool within the spool valve assembly to the deflate position. The spool remains in the deflate mode until it is volitionally shifted to the inflate position by deforming the fluid transfer bulb. In the deflate mode, fluid may return from the at least one inflatable cylinder through an unbiased return valve assembly and a spool valve assembly to the reservoir until the desired penile flaccidity is achieved. Any fluid flow from the fluid reservoir to the at least one inflatable penile cylinder closes the unbiased return valve assembly.

After the fluid transfer bulb or the deflate actuator bulb is deformed and the force to deform these bulbs is removed, the bulbs rebound to their original shape during a phase known as recovery. During bulb recovery, the unbiased inlet valve assembly opens and the unbiased exhaust valve assembly is closed and fluid is urged to refill the fluid transfer bulb due to a relatively sudden increase in volume or negative pressure within the bulb.

The IPP can be embodied as a two-piece IPP device that may include two major components in fluid communication via flexible tubing, where the at least one inflatable penile cylinder is considered a single major component, regardless of whether one or two cylinders are used. The two major components include the at least one inflatable penile cylinder and a multifunctional fluid reservoir, including a fluid reservoir with an integral fluid transfer system and deflate mechanism. Alternately, the fluid transfer bulb and deflate actuator bulb may be located remote from the multifunctional reservoir.

The IPP can also be embodied as a three-piece IPP device that includes three major components in fluid communication via flexible tubing, where the at least one inflatable penile cylinder is considered a single major component, regardless of whether one or two cylinders are used. The three major components include the at least one inflatable penile cylinder, the fluid reservoir and the fluid transfer pump with integral deflate mechanism.

The implantable IPP devices described herein utilize inflatable penile cylinders, fluid transfer systems, deflate mechanisms and fluid storage reservoirs. The fluid transfer systems transfer fluid from the fluid storage reservoirs to the inflatable penile cylinders to cause penile erections. The deflate mechanisms provide a means to evacuate the fluid from the inflatable penile cylinders routing it to the fluid reservoirs.

A fluid transfer system is positioned to provide fluid communication between a fluid reservoir and at least one inflatable penile cylinder. The fluid transfer system includes a fluid transfer bulb, an unbiased inlet valve assembly and an unbiased exhaust valve assembly between the fluid reservoir and the at least one inflatable penile cylinder; and may include a spool valve assembly positioned to provide fluid communication between the fluid transfer bulb and the at least one inflatable penile cylinder.

An IPP with a one-touch deflate mechanism is positioned to provide fluid communication between at least one inflatable penile cylinder and a fluid reservoir. The deflate mechanism may include a spool valve assembly positioned to provide fluid communication between the at least one inflatable penile cylinder and the fluid reservoir. The deflate mechanism may be actuated with an integral mechanical actuator such as a push button, or remotely actuated with pilot pressure from a deflate actuator bulb.

An IPP with a sustained deflate mechanism may be positioned to provide fluid communication between at least one inflatable penile cylinder and a fluid reservoir. Activation of the deflate mechanism mechanically disrupts the seating of both the unbiased inlet and exhaust valves to allow fluid flow from the at least one inflatable penile cylinder to the fluid reservoir. Alternately, a separate deflate valve may be used and mechanically activated to disrupt the seating of the deflate valve, allowing fluid flow from the at least one inflatable penile cylinder to the fluid reservoir. Disruption of the valve seating may result from external force causing deformation of an elastomeric valve seat or external force causing movement of a valve away from the valve seat.

When the fluid transfer system and deflate mechanism are configured for a two-piece IPP device, they may be fabricated with individual components assembled so they are in fluid communication. The unbiased inlet valve assembly, fluid transfer bulb, unbiased exhaust valve assembly, spool valve assembly, unbiased return valve assembly, and deflate actuator may be separate components that are in fluid communication via flexible tubing. The deflate mechanism may be actuated with an integral mechanical actuator such as a push button, or remotely actuated with pilot pressure from a deflate actuator bulb. The fluid transfer bulb and deflate actuator bulb may be remotely located from other components of the fluid transfer system and deflate mechanism either within or externally remote to the multifunctional reservoir.

When the fluid transfer system and deflate mechanism are configured for a three-piece IPP device, they may be fabricated as a unitary fluid transfer pump consisting of an unbiased inlet valve assembly, unbiased exhaust valve assembly, fluid transfer bulb, and a deflate mechanism. The fluid transfer system and deflate mechanism may include a spool valve assembly and deflate actuator button.

In an embodiment, the two-piece configuration features a multifunctional fluid reservoir consisting of fluid reservoir, unbiased inlet valve assembly, unbiased exhaust valve assembly, fluid transfer bulb, spool valve assembly, unbiased return valve assembly and deflate mechanism. The deflate mechanism includes a spool valve assembly having a spool that may be shifted to the cylinder deflate position either with a mechanical actuator such as a push button or hydraulically by fluid pilot pressure from a remote source such as a deflate actuator bulb. Alternately, the fluid transfer bulb and/or deflate actuator bulb may be remote from the multifunctional reservoir, in fluid communication with the fluid transfer system and deflate mechanism via flexible tubing.

Application of pressure on the ends of the spool within the spool valve assembly shifts the spool to the desired position. During cylinder inflation, fluid from the fluid transfer bulb is directed to the end of the spool that shifts the spool to the cylinder inflate position. During cylinder deflation, force from the cylinder deflate actuator is directed to the end of the spool that shifts the spool to the cylinder deflate position. The force required to shift the spool valve assembly to the cylinder deflate mode may be either a mechanical actuator button or fluid pressure from a deflate actuator bulb.

The spool valve assembly serves as a two-position directional valve without a neutral position. The spool valve assembly either routes fluid from the fluid transfer bulb to the inflatable penile cylinders in the inflate mode or conversely, from the inflatable penile cylinders to the fluid reservoir in the cylinder deflate mode. One-way unbiased check valve assemblies within the spool eliminate flow in the reverse direction. An unbiased return valve assembly prevents fluid flow from the reservoir to the inflatable penile cylinders, and an unbiased exhaust valve assembly prevents fluid flow from the inflatable penile cylinders to the fluid transfer bulb.

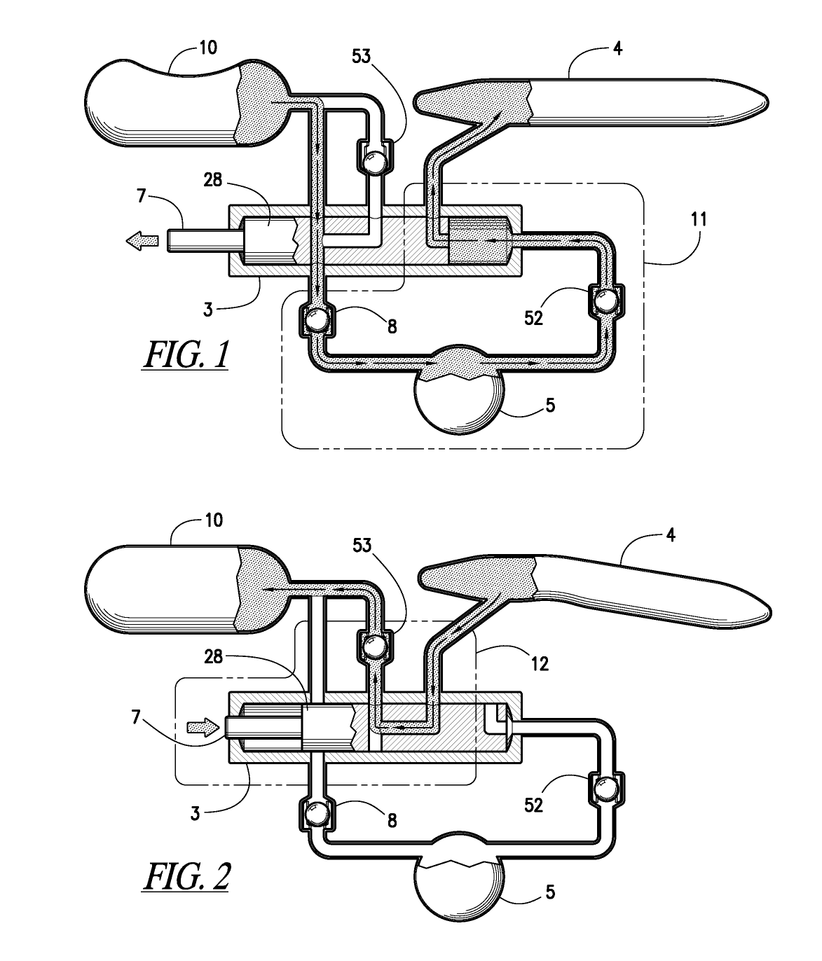

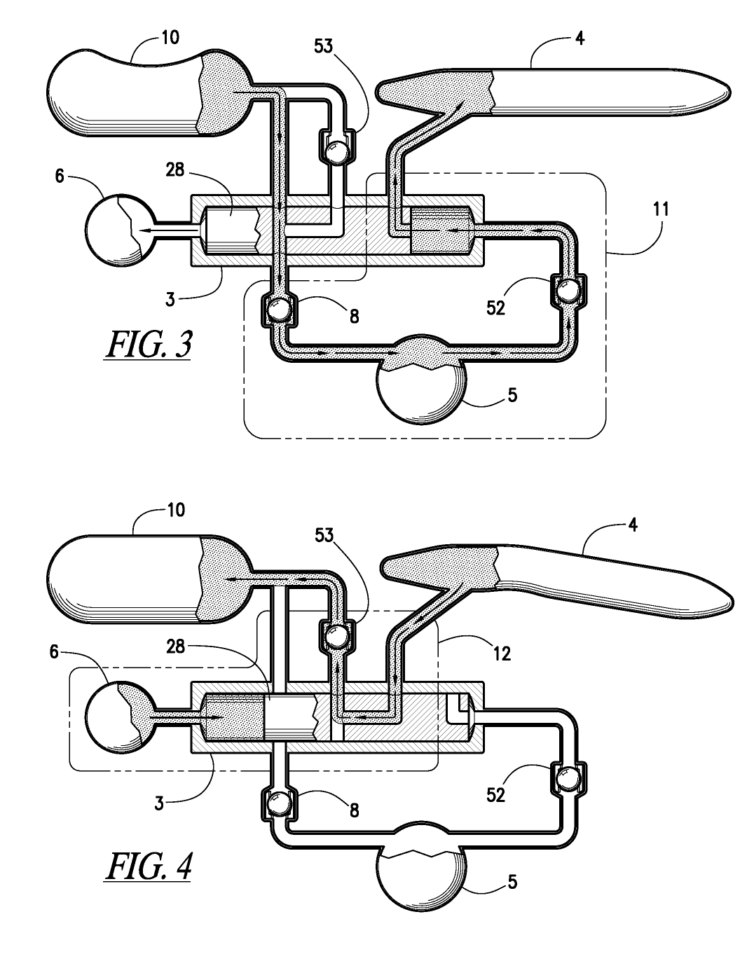

Referring now to the drawings, FIG. 1 schematically depicts an implantable IPP incorporating a spool valve assembly 3 in an example configuration, a fluid reservoir 10, at least one inflatable penile cylinder 4, a fluid transfer system 11 and a deflate mechanism. Spool valve assembly 3, including a deflate actuator button 7 and a spool 28, is depicted in the cylinder inflate mode. Fluid transfer system 11 includes the unbiased inlet valve assembly 8, fluid transfer bulb 5, an unbiased exhaust valve assembly 52 and spool valve assembly 3. This schematic depicts the fluid flow from reservoir 10 through fluid transfer system 11 to inflatable penile cylinder 4. FIG. 1 and FIG. 2 both depict unbiased inlet valve assembly 8, unbiased exhaust valve assembly 52 and unbiased return valve assembly 53 as separate components that are not integrated into other components. In an example embodiment of a two-piece IPP device, unbiased exhaust valve assembly 52 and an unbiased return valve assembly 53 are integrated in spool 28 of spool valve assembly 3. In an example embodiment of a three-piece IPP device, unbiased exhaust valve assembly 52 and unbiased return valve assembly 53 are incorporated in spool 28 of spool valve assembly 3 and unbiased inlet valve assembly 8 is incorporated with fluid transfer bulb 5 into fluid transfer pump 20 (not depicted in FIG. 1).

Spool valve assembly 3 may include a housing with cylindrical bore and fluid ports; housing end caps; spool 28 with fluid ports, fluid channels and one or more valve chambers, the spool capable of shifting axially to cooperate with fluid ports in the housing; one or more spool end caps; unbiased exhaust valve assembly 52 and unbiased return valve assembly 53. Spool valve assembly 3 acts as a two-position directional valve that is volitionally shifted to the inflate or deflate modes using forces external to spool valve assembly 3. Spool valve assembly 3 may have unbiased exhaust valve assembly and unbiased return valve assembly within spool 28 to prevent fluid backflow through spool valve assembly 3.

Inflatable penile cylinder 4 may include an elongated, hollow, elastomeric-type inflatable chamber or bladder affixed to a more rigid cylinder base, with tubing exiting the base for fluid communication with the fluid transfer system and deflate mechanism. The inflatable penile cylinder is implanted in the corpus cavernosum of the penis. The inflatable penile cylinder can have a distensible, non-distensible or limited distensibility chamber or bladder.

Unbiased exhaust valve assembly 52 is or functions as a one-way check valve located to provide fluid communication between fluid transfer bulb 5 and at least one inflatable penile cylinder 4. Unbiased exhaust valve assembly 52 prevents backflow of fluid from at least one inflatable penile cylinder 4 to fluid transfer bulb 5 when fluid transfer bulb 5 recovers from deformation. A pressure differential caused by the deformation of the fluid transfer bulb 5 opens the unbiased exhaust valve assembly 52, allowing fluid to flow from fluid transfer bulb 5 to at least one inflatable penile cylinder 4.

Unbiased return valve assembly 53 is or functions as a one-way check valve that provides fluid communication between at least one inflatable penile cylinder 4 and fluid reservoir 10. Unbiased return valve assembly 53 prevents backflow of fluid from fluid reservoir 10 to at least one inflatable penile cylinder 4. A pressure differential caused by the pressurization of the at least one inflatable penile cylinder 4 opens unbiased return valve assembly 53, allowing fluid to flow from at least one inflatable penile cylinder 4 to the fluid reservoir 10. Unbiased return valve assembly 53 can be located in a deflate mode fluid pathway within spool 28 of spool valve assembly 3.

Unbiased inlet valve assembly 8 is a one-way check valve to provide fluid communication between fluid reservoir 10 and fluid transfer bulb 5. When fluid transfer bulb 5 is deformed, it prevents backflow of fluid to fluid reservoir 10. A pressure differential caused by the recovery of fluid transfer bulb 5 acts to open unbiased inlet valve assembly 8 to admit fluid from fluid reservoir 10 to refill fluid transfer bulb 5.

Fluid transfer system 11 may include unbiased inlet valve assembly 8 located to provide fluid communication between fluid reservoir 10 and fluid transfer bulb 5, fluid transfer bulb 5 located to provide fluid communication between unbiased inlet valve assembly 8 and unbiased exhaust valve assembly 52, unbiased exhaust valve assembly 52 located to provide fluid communication between fluid transfer bulb 5 and at least one inflatable penile cylinder 4, and a spool valve assembly located to provide fluid communication between fluid transfer bulb 5 and at least one inflatable penile cylinder 4. The fluid transfer system components may be configured into a unitary fluid transfer pump component and may include a deflate mechanism. For example, a three-piece IPP device having a fluid transfer system intended for implantation in the scrotum could be configured as a unitary pump component having an integral deflate mechanism. For a two-piece IPP device, a fluid transfer system incorporated into a multifunctional reservoir could be configured with fluid transfer components in fluid communication via flexible tubing. The fluid transfer system is volitionally operated by the patient to achieve an artificial penile erection.

Fluid transfer bulb 5 may be implemented as an elastomeric bulb that can be volitionally deformed to exhaust fluid and recovers to refill with fluid when the force to deform it is removed. Fluid transfer bulb 5 may be configured as an integral part of a fluid transfer pump or an independent component of fluid transfer system 11 connected to unbiased inlet valve assembly 8 and spool valve assembly 3 with flexible tubing.

FIG. 2 is a schematic of an implantable IPP incorporating spool valve assembly 3 in one example configuration, fluid reservoir 10, at least one inflatable penile cylinder 4, fluid transfer system and deflate mechanism 12. Spool valve assembly 3, including deflate actuator button 7 and spool 28, is depicted in the cylinder deflate mode. Deflate mechanism 12 includes deflate actuator button 7, spool valve assembly 3 and unbiased return valve assembly 53. This schematic depicts the fluid flow from cylinder 4 through the deflate mechanism 12 to reservoir 10.

Deflate mechanism 12 is volitionally actuated by the patient and returns the penis to a flaccid state. While deflate mechanism 12 is depicted in FIG. 2 as including deflate actuator button 7 that applies mechanical force, the deflate mechanism may alternatively utilize a deflate actuator bulb that applies hydraulic pilot pressure to shift spool 28 within spool valve assembly 3 to the deflate position. Spool 28 within spool valve assembly 3 may include an unbiased return valve assembly that allows fluid to flow from at least one inflatable penile cylinder 4 to fluid reservoir 10 when a pressure differential between cylinder 4 and fluid reservoir 10 is greater in cylinder 4, and prevents backflow of fluid from fluid reservoir 10 to cylinder 4 when the pressure differential between cylinder 4 and fluid reservoir 10 is greater in fluid reservoir 10. The patient may reduce cylinder deflation time or achieve greater flaccidity by squeezing the penis to increase the pressure within at least one inflatable penile cylinder 4 thereby increasing the pressure differential of at least one inflatable penile cylinder 4 over fluid reservoir 10. In a two-piece IPP device, a deflate mechanism may be incorporated into a multifunctional fluid reservoir, where the spool valve assembly is contained within the reservoir. Either a deflate actuator button that is integral with spool valve assembly or a deflate actuator bulb that provides hydraulic pilot pressure and is fluidically connected to the spool valve assembly may be used to actuate the deflate mechanism. In a three-piece IPP device, the deflate mechanism may be incorporated with a fluid transfer system into a unitary fluid transfer pump implanted in the scrotum.

FIG. 3 schematically depicts an implantable IPP incorporating spool valve assembly 3 in one example configuration, fluid reservoir 10, at least one inflatable penile cylinder 4, fluid transfer system 11 and a deflate mechanism with deflate actuator bulb 6. Spool valve assembly 3 with spool 28 is depicted in the cylinder inflate mode. Fluid transfer system 11 is comprised of unbiased inlet valve assembly 8, fluid transfer bulb 5, unbiased exhaust valve assembly 52 and spool valve assembly 3. This schematic depicts the fluid flow from reservoir 10 through fluid transfer system 11 to inflatable penile cylinder 4. FIG. 3 and FIG. 4 both depict unbiased inlet valve assembly 8, unbiased exhaust valve assembly 52 and unbiased return valve assembly 53 as separate components that are not integrated into other components. In an example two-piece IPP embodiment, unbiased exhaust valve assembly 52 and unbiased return valve assembly 53 are integrated in spool 28 of spool valve assembly 3. In an example three-piece IPP embodiment, unbiased exhaust valve assembly 52 and unbiased return valve assembly 53 are integrated in spool 28 of spool valve assembly 3 and unbiased inlet valve assembly 8 is incorporated with fluid transfer bulb 5 into fluid transfer pump 20 (not depicted in FIG. 3).

FIG. 4 is a schematic of an implantable IPP incorporating spool valve assembly 3 in one example configuration, fluid reservoir 10, at least one inflatable penile cylinder 4, a fluid transfer system and deflate mechanism 12. Spool valve assembly 3 with spool 28 is depicted in the cylinder deflate mode. Deflate mechanism 12 is comprised of deflate actuator bulb 6, spool valve assembly 3 and unbiased return valve assembly 53. This schematic depicts the fluid flow from cylinder 4 through deflate mechanism 12 to fluid reservoir 10.

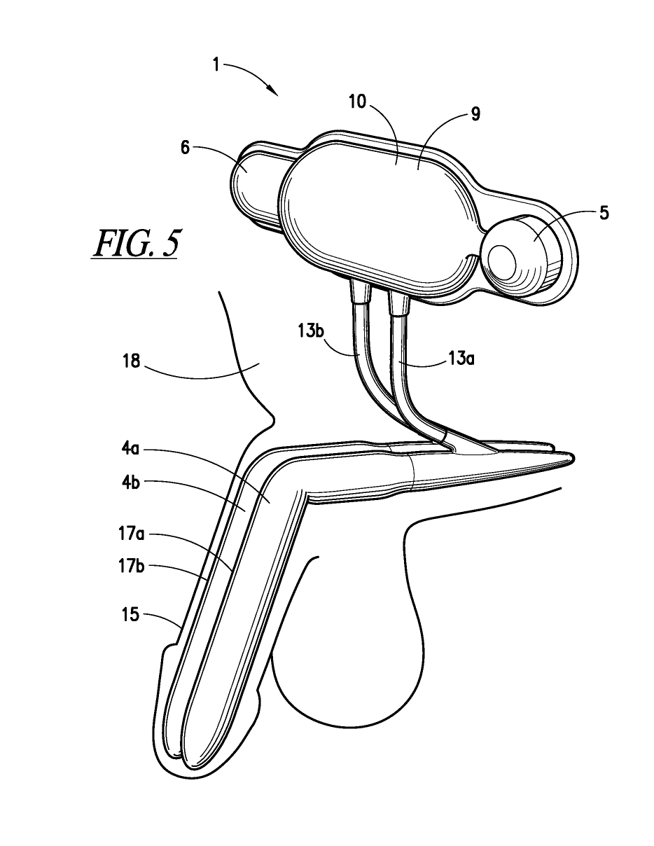

FIG. 5 depicts an example two-piece IPP 1 with penis 15 in the flaccid state. Multifunctional fluid reservoir 9 is implanted subcutaneously in lower abdomen 18. Multifunctional fluid reservoir 9 may be fabricated from elastomeric material that conforms to the implant site and has a fluid chamber that collapses as fluid is removed. Multifunctional fluid reservoir 9 can incorporate multiple components into a unitary structure and may include, for example, fluid reservoir 10, integral fluid transfer system and cylinder deflate mechanism. Flexible elastomeric tubing provides fluid communication between the reservoir, fluid transfer system and deflate mechanism components. Flexible tubing 13a provides fluid communication between inflatable penile cylinder 4a implanted in corpus cavernosum 17a of penis 15 and multifunctional reservoir 9 implanted subcutaneously in lower abdomen 18. Flexible tubing 13b provides fluid communication between inflatable penile cylinder 4b implanted in corpus cavernosum 17b of penis 15 and multifunctional fluid reservoir 9 implanted subcutaneously in lower abdomen 18. Fluid transfer bulb 5 used to operate the fluid transfer system and deflate actuator bulb 6 used to actuate the cylinder deflate mechanism are located at the lateral extremities of multifunctional fluid reservoir 9. The unbiased inlet valve assembly and spool valve assembly may be located within the reservoir.

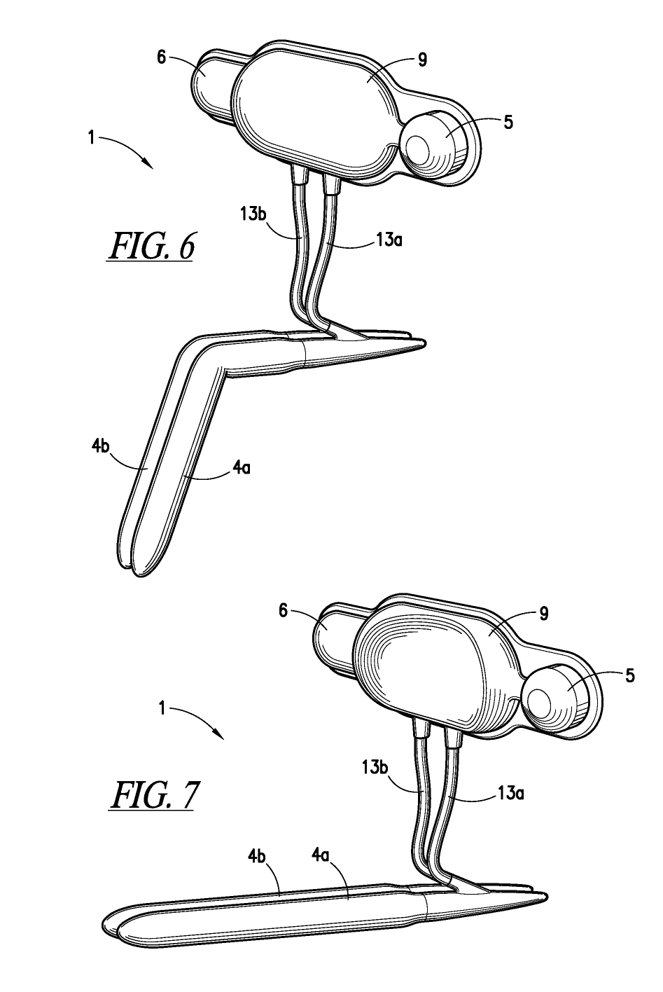

FIG. 6 depicts an example two-piece IPP 1 with multifunctional fluid reservoir 9 and penile cylinders in the flaccid state. Multifunctional fluid reservoir 9 is substantially filled with fluid while the penile cylinders are only partially filled with fluid. Fluid transfer bulb 5 operates the fluid transfer system and deflate actuator bulb 6 actuates the cylinder deflate mechanism. Flexible tubing 13a and 13b provide fluid communication between the inflatable penile cylinders and multifunctional fluid reservoir 9.

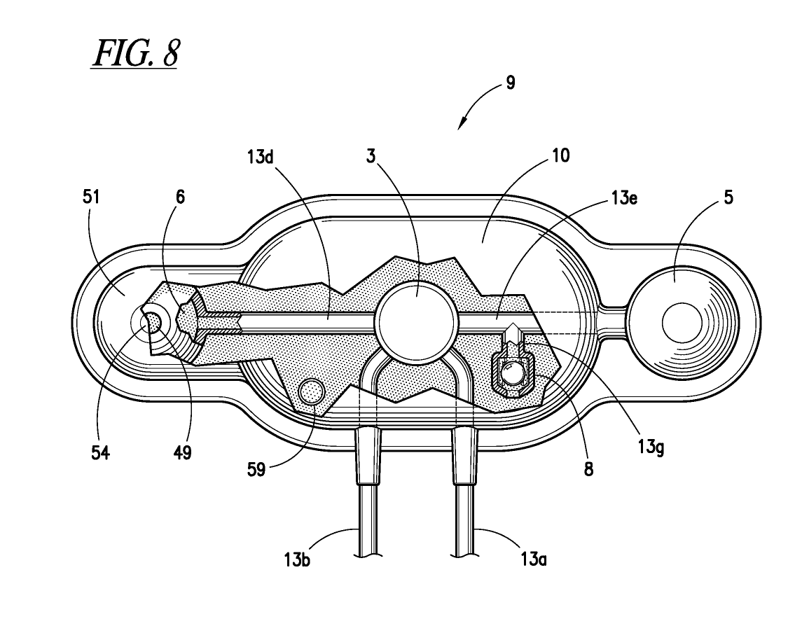

FIG. 7 depicts an example two-piece IPP 1 with multifunctional fluid reservoir 9 and penile cylinders 4a and 4b in the erectile state. Multifunctional fluid reservoir 9 is partially filled with fluid while inflatable penile cylinders 4a and 4b are substantially filled with fluid.

FIG. 8 depicts an example multifunctional fluid reservoir 9 as depicted in FIGS. 6 and 7 with fluid reservoir 10 with anterior wall 51 opened to expose fluid fill port 59, unbiased inlet valve assembly 8, spool valve assembly 3, fluid transfer bulb 5, deflate actuator bulb 6, and flexible tubing 13a, 13b, 13d, 13e and 13g. Deflate actuator bulb 6 has an aperture 49 through the apex of its dome that interacts with anterior wall 51 to comprise aperture valve assembly 54. Fluid fill port 59 is used to fill and adjust device fluid volume either at the manufacturer, if the device is prefilled, or during surgical implantation. Fluid fill port 59 can be an injection-type port where the lumen or fluid chamber of reservoir 10 may be accessed via a sharp needle. An injection-type port has an elastomeric shell incorporating a plastic or metal needle guard to prevent punctures of the reservoir shell or base and a cavity filled with a self-sealing elastomer. Alternately, fluid fill port 59 may be an elastomeric valve port that accommodates a stylus that opens the valve when inserted and closes the valve when withdrawn. Fluid fill port 59 is bonded to either the shell or base and is located where it will be convenient to remove air from fluid reservoir 10.

Fluid reservoir 10 can be used to store fluid for the IPP and would typically be implanted abdominally in the Space of Retzius or subcutaneously in the lower abdomen. Fluid reservoir 10 has an elastomeric bladder that collapses or deforms when fluid is withdrawn and returns to its normal shape as it fills with fluid. If fluid reservoir 10 is a standalone component, then it can have or be connected to a flexible tubing to provide fluid communication between it and the fluid transfer system, deflate mechanism and cylinders.

In one embodiment, a two-position spool valve assembly has a ported housing with a sliding spool, the sliding spool having fluid pathways that cooperate with housing ports to route fluid for inflating or evacuating inflatable penile cylinders. The spool has an internal unbiased exhaust valve chamber ported to provide fluid communication with the fluid transfer system, and the spool has an internal unbiased return valve chamber ported to provide fluid communication with a deflate mechanism. Incorporating the unbiased exhaust valve assembly and unbiased return valve assembly within the spool simplifies the device.

FIG. 9 is a sectional view of spool valve assembly 3 used with deflate actuator bulb 6 depicted in FIG. 8, with spool valve assembly 3 in the cylinder deflate mode. Spool valve assembly 3 includes valve housing 21; housing deflate end cap 22; housing inflate end cap 23; end cap screws 25; spool 28; spool deflate end cap 29; spool inflate end cap 30; 0-rings 31, 32, and 33; balls 35 and 36; valve seats 39 and 40; valve seat retainer 41; cylinder spigots 42 and 43; transfer bulb spigot 44; deflate bulb spigot 47; and reservoir port 48.

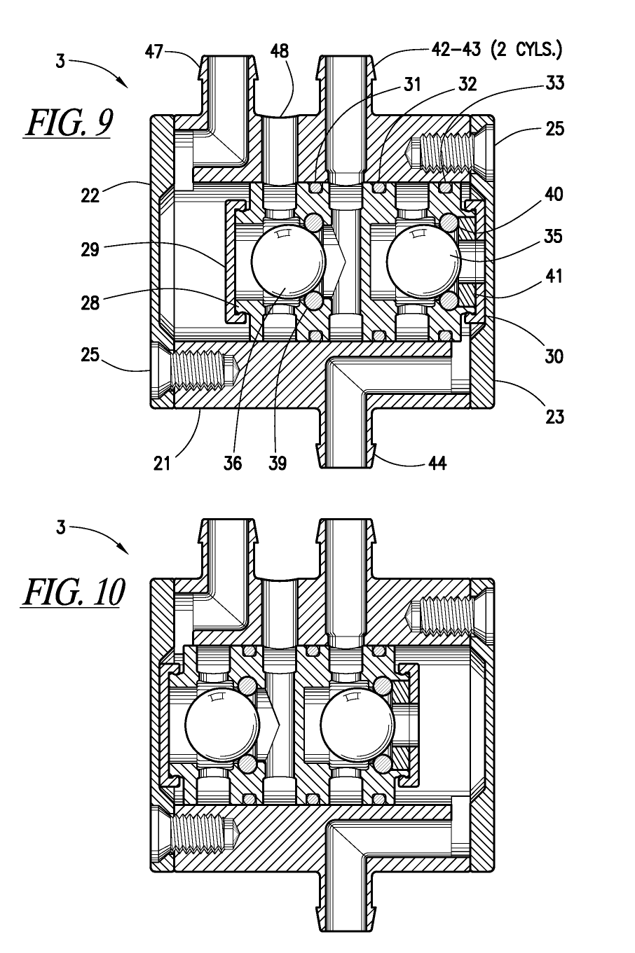

FIG. 10 is a sectional view of the spool valve assembly 3 used with deflate actuator bulb 6 depicted in FIG. 8 with spool valve assembly 3 in the inflate mode.

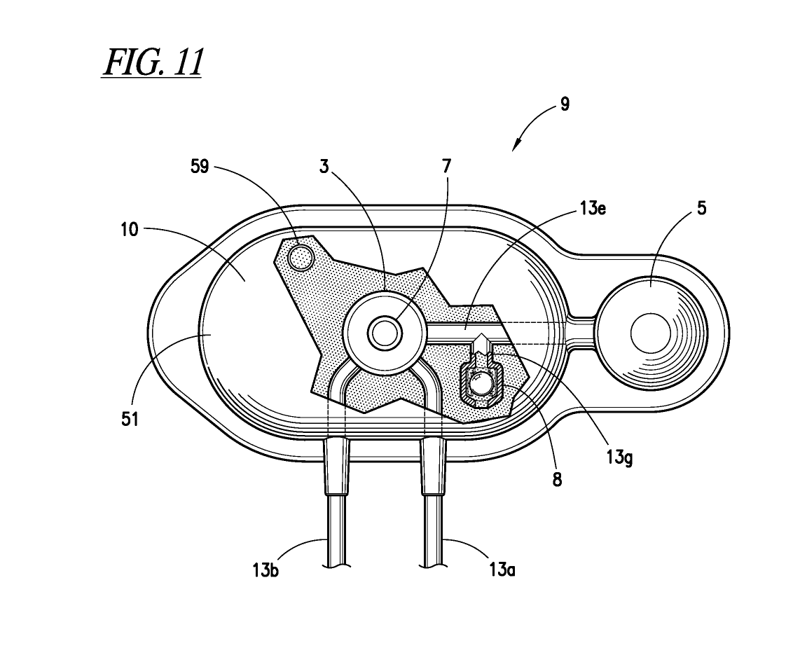

FIG. 11 depicts multifunctional reservoir 9 with fluid reservoir 10 with anterior wall 51 opened to expose fluid fill port 59, unbiased inlet valve assembly 8, spool valve assembly 3, fluid transfer bulb 5, deflate actuator button 7 and flexible tubing 13a, 13b, 13e and 13g.

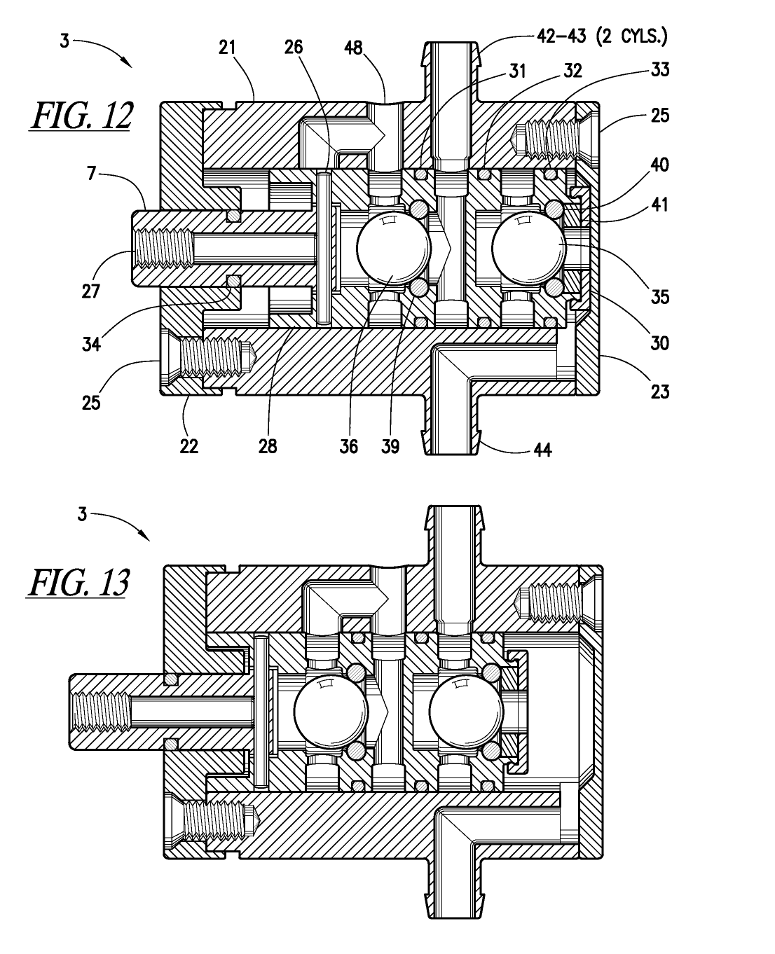

FIG. 12 is a sectional view of spool valve assembly 3 used with deflate actuator button 7 depicted in FIG. 11, with spool valve assembly 3 in the cylinder deflate mode. Spool valve assembly 3 includes valve housing 21; housing deflate end cap 22; housing inflate end cap 23; end cap screws 25; spool 28; spool inflate end cap 30; O-rings 31, 32, 33, and 34; balls 35 and 36; valve seats 39 and 40; valve seat retainer 41; cylinder spigots 42 and 43; transfer bulb spigot 44; reservoir port 48; deflate actuator button 7; deflate button pin 26; and deflate button screw 27.

FIG. 13 is a sectional view of spool valve assembly 3 used with deflate actuator button 7 depicted in FIG. 11, with spool valve assembly 3 in the inflate mode.

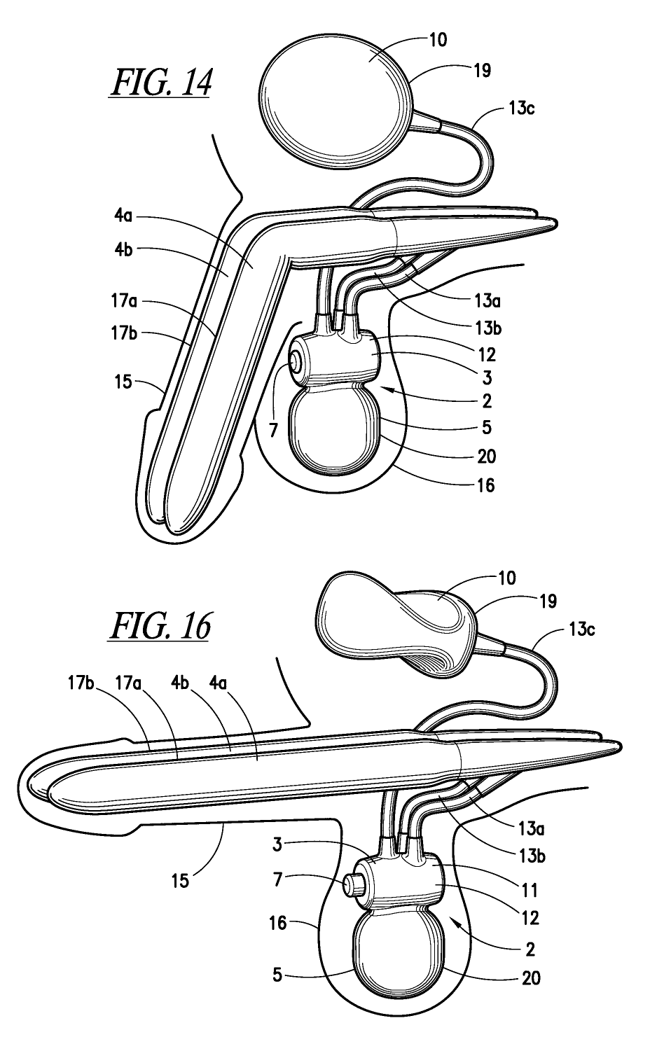

FIG. 14 depicts an implanted three-piece IPP device 2 in the flaccid state. Fluid reservoir 10 is implanted in abdominal Space of Retzius 19. Fluid transfer pump 20 with spool valve assembly 3 and integral deflate mechanism 12, including deflate actuator button 7, is implanted in scrotum 16. Inflatable penile cylinders 4a and 4b are implanted in the corpus cavernosa 17a and 17b, respectively, of penis 15. Flexible tubing 13a and 13b fluidically connect penile cylinders 4a and 4b respectively and fluid transfer pump 20. Flexible tubing 13c connects fluid reservoir 10 and fluid transfer pump 20. FIG. 14 depicts spool valve assembly 3 configured with its central axis perpendicular to the central axis of fluid transfer bulb 5.

The fluid transfer pump 20 may include a fluid transfer system and deflate mechanism that are configured into a single fluid transfer pump with the fluid transfer system including an unbiased inlet valve assembly, fluid transfer bulb, unbiased exhaust valve assembly and spool valve assembly and deflate mechanism including a deflate button actuator, spool valve assembly and unbiased return valve assembly.

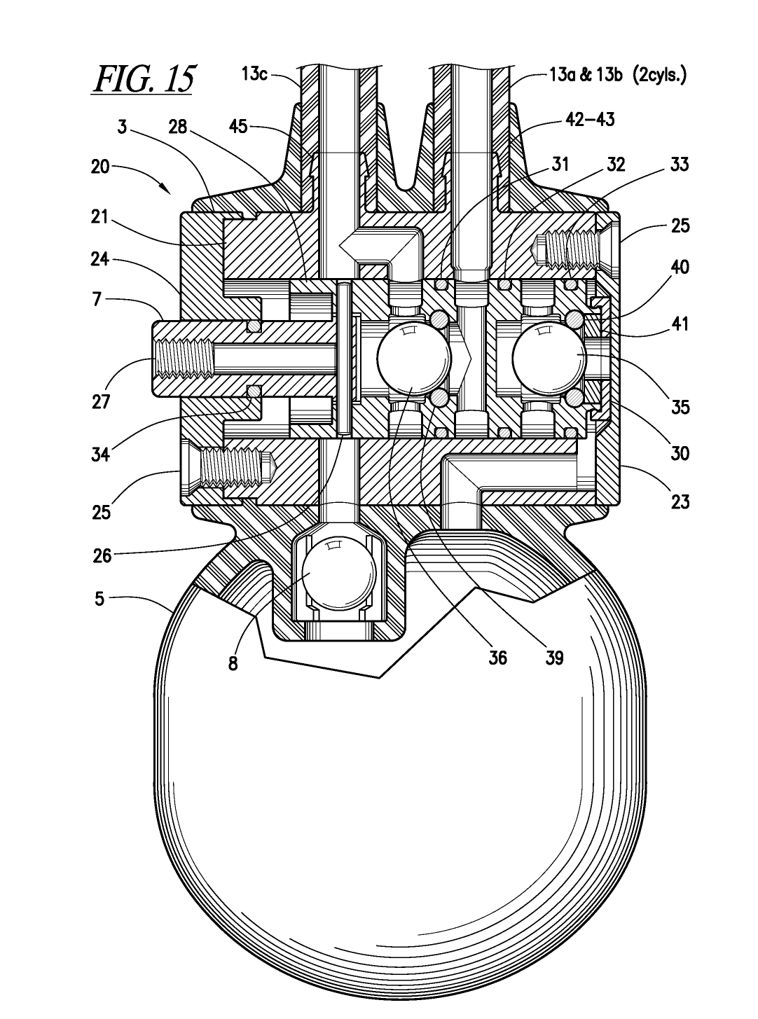

FIG. 15 is a sectional view of fluid transfer pump 20 with fluid transfer bulb 5, unbiased inlet valve assembly 8, integral deflate mechanism 12 (illustrated in FIG. 2) with spool valve assembly 3 in the cylinder deflate mode as depicted in FIG. 14. Spool valve assembly 3 includes valve housing 21; housing deflate end cap 24; housing inflate end cap 23; end cap screws 25; deflate actuator button 7; deflate button screw 27; deflate button pin 26; spool 28; spool inflate end cap 30; 0-rings 31, 32, 33, and 34; balls 35 and 36; valve seats 39 and 40; valve seat retainer 41; cylinder spigots 42 and 43; and reservoir spigot 45.

FIG. 16 depicts an example implanted three-piece piece IPP device 2 in the erectile state. Fluid reservoir 10 is implanted in the abdominal Space of Retzius 19. Fluid transfer pump 20 with spool valve assembly 3 includes fluid transfer system 11, integral deflate mechanism 12 with deflate actuator button 7, that is implanted in scrotum 16. Inflatable penile cylinders 4a and 4b are implanted in the corpus cavernosa 17a and 17b, respectively, of the penis 15. Flexible tubing 13a and 13b provide fluid communication between penile cylinders 4a and 4b, respectively, and fluid transfer pump 20. Flexible tubing 13c connects fluid reservoir 10 and fluid transfer pump 20.

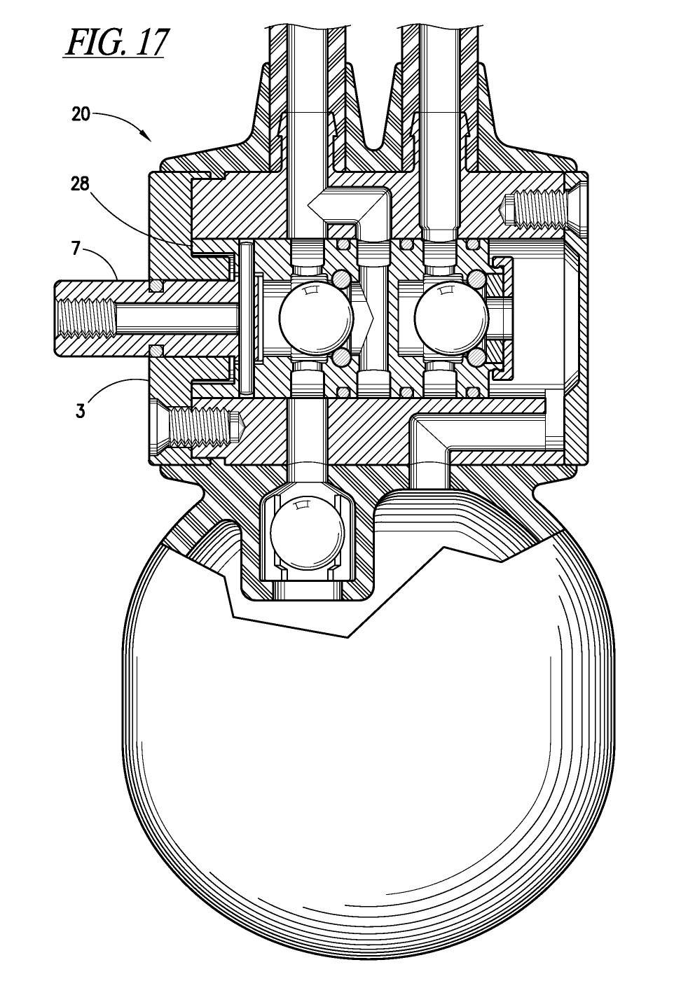

FIG. 17 is a sectional view of the fluid transfer pump 20 as depicted in FIG. 16 with spool valve assembly 3 in the cylinder inflate mode.

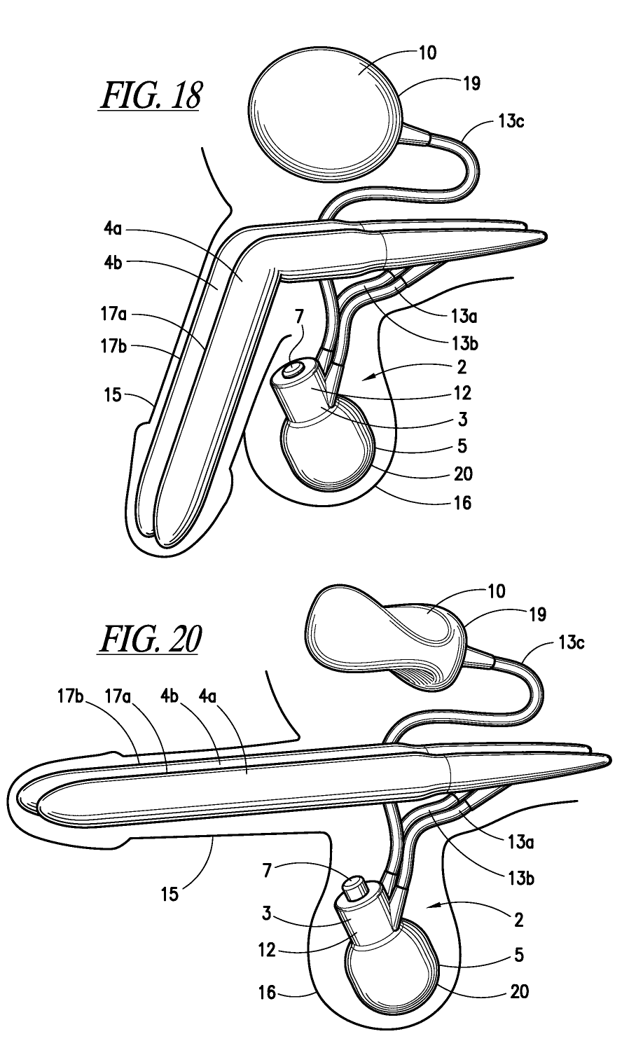

FIG. 18 depicts an example implanted three-piece IPP device 2 in the flaccid state. Fluid reservoir 10 is implanted in abdominal Space of Retzius 19. Fluid transfer pump 20 with spool valve assembly 3 and integral deflate mechanism 12, including deflate actuator button 7, is implanted in scrotum 16. Inflatable penile cylinders 4a and 4b are implanted in corpus cavernosa 17a and 17b, respectively, of penis 15. Flexible tubing 13a and 13b provide fluid communication between penile cylinders 4a and 4b, respectively, and fluid transfer pump 20. Flexible tubing 13c connects fluid reservoir 10 and fluid transfer pump 20. FIG. 18 depicts spool valve assembly 3 configured with its central axis corresponding to the central axis of fluid transfer bulb 5.

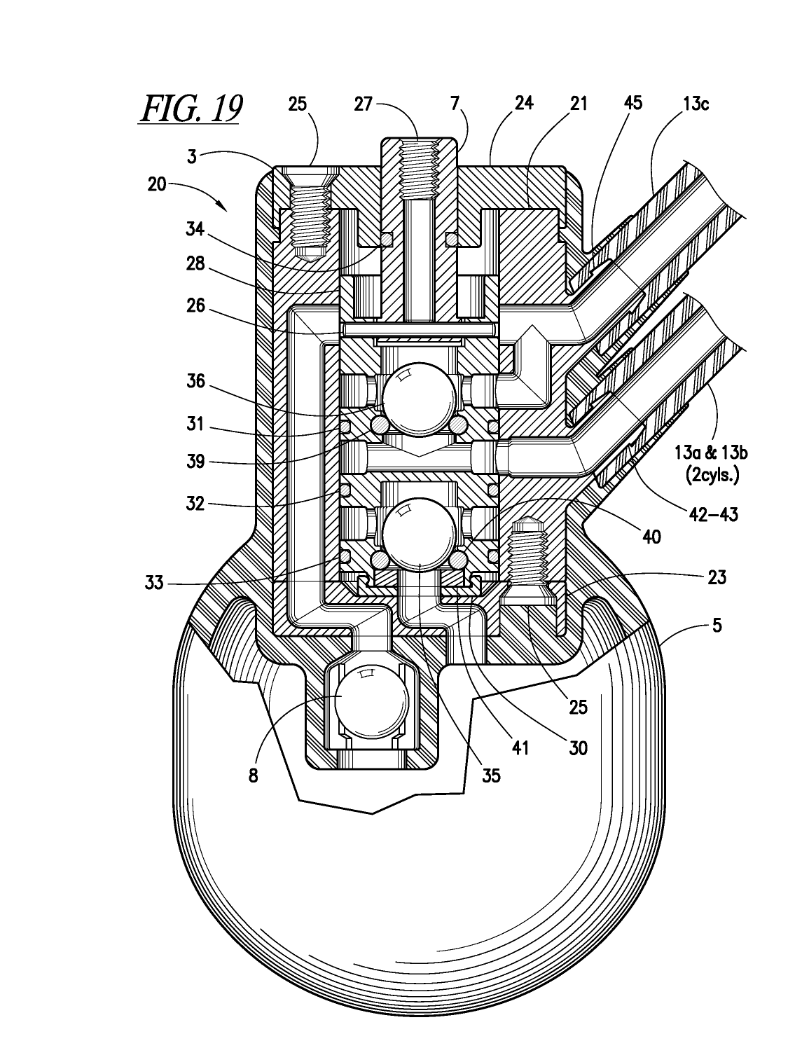

FIG. 19 is a sectional view of fluid transfer pump 20 with unbiased inlet valve assembly 8, integral deflate mechanism 12 (depicted in FIG. 2) with spool valve assembly 3 in the cylinder deflate mode as depicted in FIG. 18. Spool valve assembly 3 includes valve housing 21; housing deflate end cap 24; housing inflate end cap 23; end cap screws 25; deflate actuator button 7; deflate button screw 27; deflate button pin 26; spool 28; spool inflate end cap 30; 0-rings 31, 32, 33, and 34; balls 35 and 36; valve seats 39 and 40; valve seat retainer 41; cylinder spigots 42 and 43; and reservoir spigot 45.

FIG. 20 depicts an example implanted three-piece IPP device 2 in the erectile state. Fluid reservoir 10 is implanted in abdominal Space of Retzius 19. Fluid transfer pump 20 with spool valve assembly 3 and integral deflate mechanism 12, including deflate actuator button 7, is implanted in the scrotum 16. Inflatable penile cylinders 4a and 4b are implanted in the corpus cavernosa 17a and 17b, respectively, of penis 15. Flexible tubing 13a and 13b provide fluid communication between penile cylinders 4a and 4b, respectively, and fluid transfer pump 20. Flexible tubing 13c connects fluid reservoir 10 and fluid transfer pump 20. FIG. 20 depicts spool valve assembly 3 configured with its central axis corresponding to the central axis of fluid transfer bulb 5.

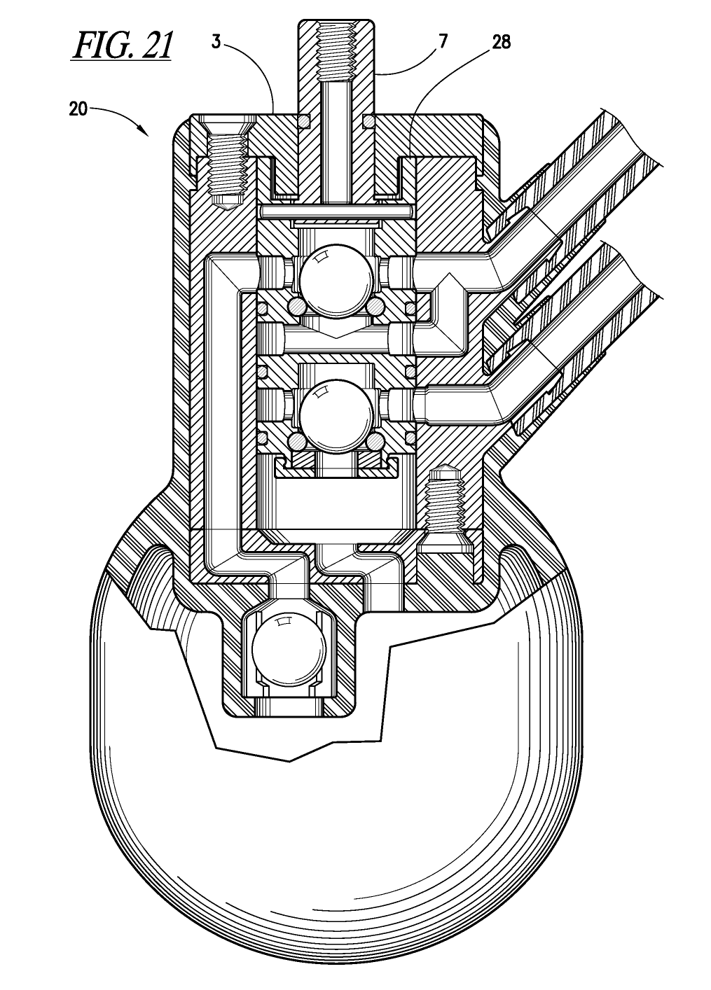

FIG. 21 is a sectional view of the fluid transfer pump 20 depicted in FIG. 20 with spool valve assembly 3 in the cylinder inflate mode.

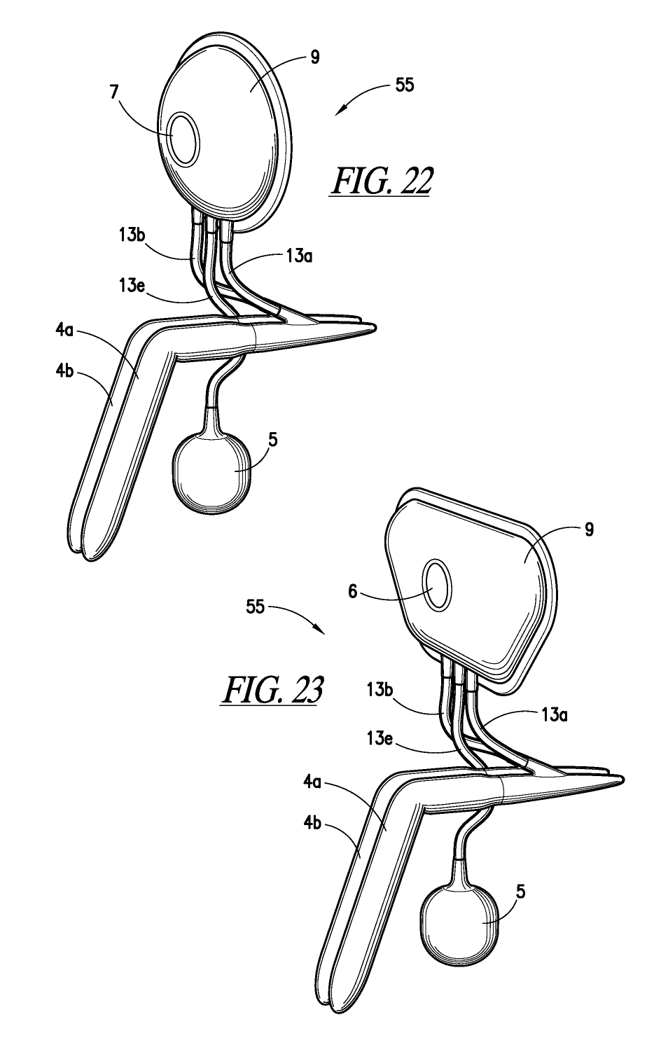

FIG. 22 depicts three-piece IPP 55 of the present disclosure with substantially round multifunctional fluid reservoir 9, cylinders 4a and 4b in the flaccid state, and fluid transfer bulb 5 in fluid communication via flexible tubing 13e with other components (not depicted) comprising the fluid transfer system that are contained within multifunctional reservoir 9. Fluid transfer bulb 5 operates the fluid transfer system and deflate actuator button 7 actuates the cylinder deflate mechanism (not depicted). Flexible tubing 13a and 13b provide fluid communication between inflatable penile cylinders 4a and 4b, respectively, to multifunctional reservoir 9.

FIG. 23 depicts an example three-piece IPP 55 device with substantially trapezoidal multifunctional fluid reservoir 9, cylinders 4a and 4b in the flaccid state, and fluid transfer bulb 5 in fluid communication via flexible tubing 13e with other components (not depicted) comprising the fluid transfer system that are contained within multifunctional reservoir 9. Fluid transfer bulb 5 operates the fluid transfer system and deflate actuator bulb 6 actuates the cylinder deflate mechanism (not depicted). Flexible tubing 13a and 13b provide fluid communication between inflatable penile cylinders 4a and 4b, respectively and multifunctional reservoir 9.

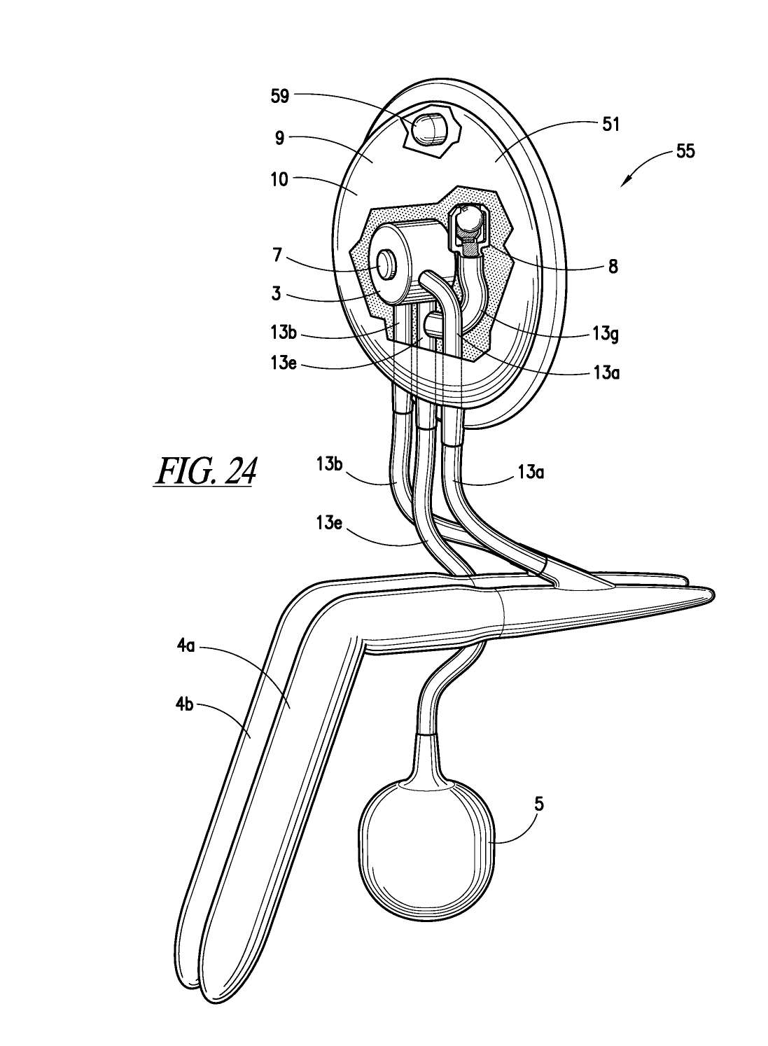

FIG. 24 depicts the IPP 55 depicted in FIG. 22 with multifunctional reservoir 9 including fluid reservoir 10 with anterior wall 51 opened to expose unbiased inlet valve assembly 8; spool valve assembly 3; deflate actuator button 7; fluid fill port 59; and flexible tubing 13a, 13b, 13e, and 13g. Tubing 13e provides fluid communication between spool valve assembly 3 and fluid transfer bulb 5. Fluid transfer bulb 5 can be remotely located in the scrotum or abdomen. Flexible tubing 13a and 13b provide fluid communication between spool valve assembly 3 and inflatable penile cylinders 4a and 4b, respectively. Spool valve assembly 3 is depicted in and described in connection with FIG. 12 (deflate mode) and FIG. 13 (inflate mode).

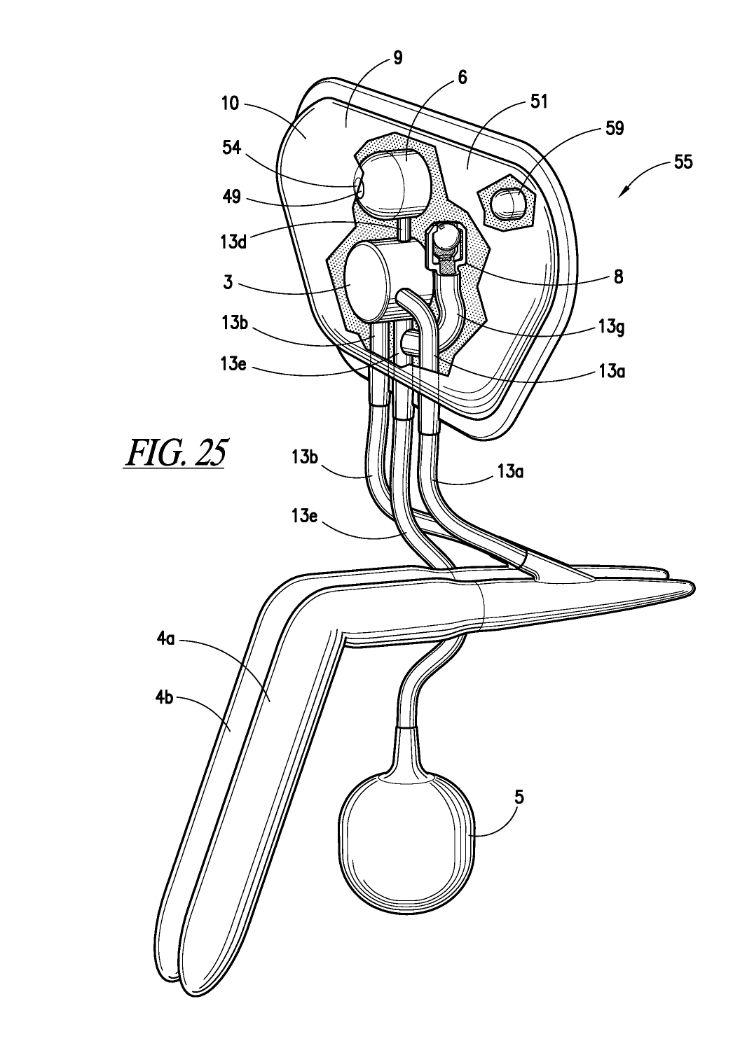

FIG. 25 depicts IPP 55 as depicted in FIG. 23 with multifunctional reservoir 9 including fluid reservoir 10 with anterior wall 51 opened to expose unbiased inlet valve assembly 8; spool valve assembly 3; deflate actuator bulb 6; fluid fill port 59; and flexible tubing 13a, 13b, 13d, 13e and 13g. Deflate actuator bulb 6 has aperture 49 through the apex of its dome that interacts with anterior wall 51 to comprise aperture valve 54. Flexible tubing 13a and 13b provide fluid communication between spool valve assembly 3 and inflatable penile cylinders 4a and 4b respectively. Spool valve assembly 3 is depicted in and described in connection with FIG. 9 (deflate mode) and FIG. 10 (inflate mode).

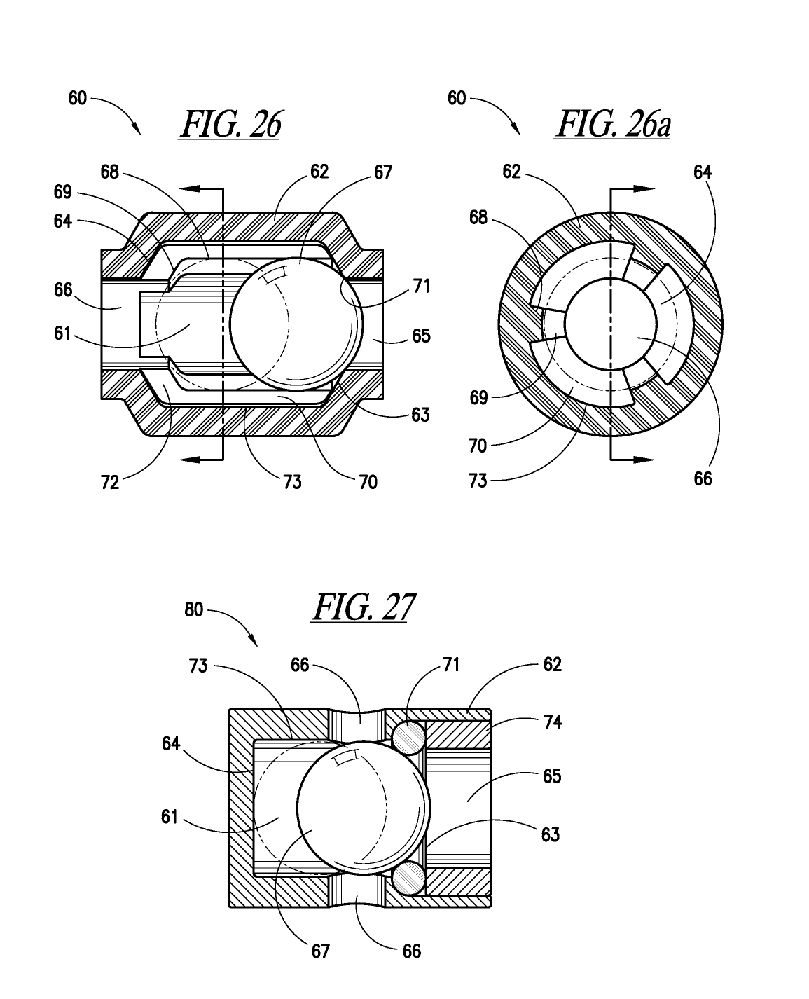

FIG. 26 depicts an unbiased check valve assembly of the type depicted for the unbiased inlet valve assembly throughout the present disclosure. FIG. 26a is a cross-sectional view of the chamber of the valve in FIG. 26 depicting the chamber end with exit port. Unbiased check valve assembly 60 is configured with valve chamber 61 having entry port 65 on valve chamber end 63 and exit port 66 on valve chamber end 64 opposite entry port 65 and valve chamber wall interior surface 73 between chamber ends 63 and 64. Valve seat 71 is formed at the junction where chamber end 63 and entry port 65 connect. Unbiased check valve assembly 60 has an elastomeric chamber wall 62 so valve seat 71 may be molded integral with chamber wall 62. The surface of chamber end 64 containing exit port 66 is irregular to prevent valve 67 from seating against exit port 66, allowing fluid to flow around valve 67 and through exit port 66. Radial ribs 69 emanating from chamber end 64 provide an irregular surface to permit fluid flow around valve 67 in the radial flow channels 72 between radial ribs 69 and chamber end 64. Axial ribs 68 emanating from valve chamber wall interior surface 73 keep valve 67 in the center of valve chamber 61 and axial flow channels 70 between axial ribs 68 and interior surface 73 allow fluid flow around valve 67.

FIG. 27 depicts unbiased check valve assembly 80 of the type depicted for the unbiased exhaust and return valve assembles contained in spool valve assembly 3 on FIGS. 9, 10, 12, 13, 15, 17, 19, and 21 of the present disclosure. Unbiased check valve assembly 80 is configured with valve chamber 61 having entry port 65 on valve chamber end 63 and chamber wall interior surface 73 between chamber ends 63 and 64. At least one exit port 66 penetrates valve chamber wall 62. Unbiased check valve assembly 80 has a valve chamber wall 62 fabricated from a substantially rigid material with an elastomeric valve seat 71 between valve chamber interior wall 73 and chamber end 63. With substantially rigid chamber wall 62, valve 67 and elastomeric valve seat 71 may be installed through entry port 65 and secured with valve seat retainer 74. Alternately, chamber end 64 could be a separate component and valve seat 71 and valve 67 could be installed through the end opposite entry port 65 and retained with chamber end 64.

The Figures illustrate a spool valve assembly that is designed for repeated assembly and disassembly for developmental purposes. Screws 25 used to retain end caps 22, 23, and 24 to housing 21 are convenient for developmental purposes, but would be an unlikely choice in the manufactured version. Instead, end caps 22, 23, and 24 might be secured to housing 21 by welding, bonding, snap fitting, or press fitting. Valve seat retainer 41 could be omitted in a manufactured version. O-rings 31, 32, 33, and 34 might be omitted or replaced with wiper-type elements integrally molded onto the major diameter of a plastic spool or deflate actuator button. Deflate button pin 26 and deflate button screw 27, associated with deflate actuator button 7, are convenient for developmental efforts, but would likely be omitted in favor of alternative methods such as snap fit.

Materials for fabrication of the spool valve assembly components are preferably biocompatible and rigid metals or plastics that will remain chemically and dimensionally stable when exposed to the hostile environment of human implantation. Biostable candidate metal materials for the housing, housing end caps, spool, spool end caps and spigots include a cobalt, nickel, chromium, molybdenum alloy known as MP35N or titanium alloy. Biostable candidate plastic materials for the housing, housing end caps, spool, spool end caps, spigots and deflate actuator button include polypropylene, polysulfone, polyetheretherketone (PEEK) and acrylic. Candidate elastomeric materials for the o-rings and valve assembly seats include silicone and VITON® brand fluoroelastomer, commercially available from DuPont Performance Elastomers of Wilmington, Del.

According to an embodiment, an unbiased one-way check valve assembly can be accomplished with a valve component having a specific gravity substantially similar to the fluid used to operate the IPP device. Materials for unbiased check valve, such as balls or poppets, are preferably biocompatible plastics that will remain chemically and dimensionally stable when exposed to the hostile environment of human implantation. Additionally, it is desirable that the specific gravity of the unbiased valve material be substantially similar to the specific gravity of the fluid used to operate the IPP. For example, polypropylene, having a specific gravity of approximately 0.9, when used with an IPP device filled with normal saline solution having a specific gravity of approximately 1.0, moves with the fluid to allow the valve to open and close freely. Selection of a valve material for an unbiased check valve assembly that moves freely with the flow of fluid eliminates the need for a spring to bias the check valve in a closed position. Historically, one-way check valve assembles used with IPP devices used a metal valve and relied on a spring to bias the valve in a closed position. Metal valves have a much higher specific gravity (e.g., the specific gravity of titanium is approximately 4.5, the specific gravity of MP35N is approximately 8.0, and the specific gravity of stainless steel is typically 7.6-7.9). Even Teflon valves used in industrial applications for chemical resistance have a specific gravity of 2.1 or greater. Besides overcoming spring bias, the fluid flow has to move the mass of the valve component. Elimination of a spring and the bias it imposes on the valve eliminates the resistance to valve opening. With the inlet valve on an IPP device, the negative force to draw fluid from the reservoir through the inlet valve is significantly reduced and the flow rate is increased with an unbiased check valve. Similarly, the resistance to pumping force is reduced and fluid flow is increased with an unbiased exhaust valve. An unbiased return valve opens easier and allows for faster deflation of the inflatable penile cylinders.

Elastomeric O-rings or integrally molded wipers on a spool can be used to achieve a fluid seal between the spool and spool valve housing or create resistance to movement of the spool within the housing. Similarly, elastomeric O-rings or integrally molded wipers on the deflate actuator button can be used to achieve a fluid seal between the deflate actuator button and the spool valve housing or create resistance to movement of the deflate actuator button within the housing.

The present disclosure teaches providing an IPP device comprising a fluid reservoir, at least one inflatable penile cylinder, a fluid transfer system in fluid communication with said fluid reservoir and said at least one inflatable penile cylinder to inflate said at least one penile cylinder with fluid from said fluid reservoir, a deflate mechanism in fluid communication with said fluid reservoir and said at least one inflatable penile cylinder to return fluid from said at least one inflatable penile cylinder to said fluid reservoir and a plurality of flexible tubing providing fluid communication between the components. The fluid transfer system includes an unbiased inlet valve assembly in fluid communication with said fluid reservoir; a fluid transfer bulb in fluid communication with said unbiased inlet valve assembly; and an unbiased exhaust valve assembly in fluid communication with said fluid transfer bulb and said at least one inflatable penile cylinder. The fluid transfer system is volitionally activated to transfer fluid from said fluid reservoir to said at least one inflatable penile cylinder. The fluid transfer system may include a spool valve assembly in fluid communication with the fluid transfer bulb and the at least one inflatable penile cylinder. The deflate mechanism may include a spool valve assembly in fluid communication with the fluid reservoir and the at least one inflatable penile cylinder and an unbiased return valve assembly in fluid communication with the reservoir and the at least one inflatable penile cylinder, and a deflate actuator to mechanically or hydraulically shift the spool valve assembly. The deflate mechanism may include an unbiased return valve assembly in fluid communication with said fluid reservoir and said at least one inflatable penile cylinder. The fluid transfer system is positioned to provide fluid communication between the fluid reservoir and the at least one inflatable penile cylinder. The deflate mechanism is positioned to provide fluid communication between the fluid reservoir and the at least one inflatable penile cylinder. The fluid transfer system and the deflate mechanism may include a shared or common spool valve assembly.

One way check valve assemblies may be used independently or in conjunction with other valve assemblies such as spool valve assemblies. The spool valve assembly acts as a two-position directional valve that is volitionally shifted between the cylinder inflate and cylinder deflate modes using forces external to the spool valve assembly. The spool valve assembly has a spool that may include an unbiased exhaust valve assembly and an unbiased return valve assembly. The unbiased exhaust valve assembly comprises a valve chamber, a valve component such as a ball or poppet, a valve seat, entry and exit ports. Similarly, the unbiased return valve assembly comprises a valve chamber, a valve such as a ball or poppet, a valve seat, entry and exit ports. The spool valve assembly is a two-position directional valve that can be shifted to the inflate mode with hydraulic pilot pressure from a fluid transfer bulb directed at the end of the spool that is ported to inflate at least the one inflatable penile cylinder. Alternately, the spool valve assembly can be shifted to the deflate mode with mechanical force from a deflate actuator button directed at the end of the spool that is ported to deflate the at least one inflatable penile cylinder. The spool valve assembly with the deflate actuator button that mechanically shifts the spool within the spool valve assembly to the deflate mode can have the deflate actuator button and the spool configured to provide fluid communication between a fluid reservoir and a headspace between the spool valve housing and the end of the spool ported to deflate the at least one inflatable cylinder. The headspace fluid communication with the fluid reservoir permits fluid to exit the headspace when the spool shifts to the inflate mode and enter the headspace when the spool shifts to the deflate mode, thus minimizing any resistance to spool movement.

When a one-way check valve assembly is used independently, the assembly is comprised of a valve chamber, a valve component such as a ball or poppet, a valve seat, and entry and exit ports.

According to another embodiment, a two-piece IPP device includes a multifunctional fluid reservoir and at least one inflatable penile cylinder and flexible tubing connecting these components. The multifunctional fluid reservoir includes a fluid transfer system to inflate the at least one penile cylinder with fluid from a fluid reservoir, and a cylinder deflate mechanism to return fluid from the at least one inflatable penile cylinder to the fluid reservoir. The fluid transfer system comprises an unbiased inlet valve assembly in fluid communication with the fluid reservoir, a fluid transfer bulb in fluid communication with the unbiased inlet valve assembly, an unbiased exhaust valve assembly in fluid communication with the fluid transfer bulb, and at least one inflatable penile cylinder. The fluid transfer system may include a spool valve assembly in fluid communication with the fluid transfer bulb and the at least one inflatable penile cylinder. The deflate mechanism may include a spool valve assembly in fluid communication with the fluid reservoir and the at least one inflatable penile cylinder, an unbiased return valve assembly in fluid communication with the reservoir and the at least one inflatable penile cylinder, and a deflate actuator to mechanically or hydraulically shift the spool valve assembly. The spool valve assembly functions as a two-position directional valve assembly that is volitionally shifted to the cylinder inflate mode or the cylinder deflate mode using external force applied to the spool within the spool valve assembly.

In one embodiment, a multifunctional reservoir has a fluid reservoir segment with a substantially flat posterior wall or base and a curved anterior wall with a hollow space between the posterior wall and the anterior wall functioning as a fluid reservoir. One side of the base extends laterally beyond the fluid reservoir segment serving as a base for a fluid transfer bulb.

In another embodiment a multifunctional reservoir has a fluid reservoir segment with a substantially flat posterior wall or base and a curved anterior wall with a hollow space between the posterior and the anterior walls functioning as a fluid reservoir. The base extends laterally on both sides of the fluid reservoir segment serving as a base for a fluid transfer bulb on one side and a deflate actuator bulb on the opposite side. The deflate actuator bulb is surrounded by a lower profile extension of the anterior wall of the fluid reservoir, laterally extending the fluid reservoir to supply fluid to the deflate actuator bulb. The anterior wall surrounding the deflate actuator bulb cooperates with an aperture at the apex of the deflate actuator bulb covering the aperture to serve as an aperture valve. When the patient deforms the deflate actuator bulb, the anterior wall contacts the aperture to close the aperture valve. The deflate bulb actuator is volitionally deformed, causing fluid to be directed as pilot pressure to the end of a spool that is ported to deflate the at least one inflatable penile cylinder, thereby shifting the spool within the spool valve assembly actuating the deflate mechanism. The aperture valve opens in the absence of external force on the deflate actuator bulb so that the recovery of the deflate actuator bulb urges fluid to refill the deflate actuator bulb through the aperture valve. The aperture valve remains open to vent fluid from the spool valve assembly, allowing the spool to shift upon activation of the inflate mode.

In any of a variety of embodiments, a multifunctional fluid reservoir can have a fluid reservoir segment with an elongated shape. The elongated fluid reservoir segment can be between 1.5 and 3.0 inches wide and between 4.0 and 6.0 inches long. The multifunctional reservoir can have an overall width between 1.5 and 4.0 inches and an overall length between 5.0 and 11 inches. The fluid reservoir can have a fluid volume ranging from 50 to 150 cubic centimeters. It will be appreciated by those of ordinary skill in the art that these dimensions and volumes are provided for illustrative purposes only and are not intended to limit the scope of the present invention.

In another embodiment, a multifunctional reservoir has a fluid reservoir segment with a substantially flat posterior wall or base and substantially flat anterior wall with a hollow space between the posterior and the anterior walls functioning as a fluid reservoir. A fluid transfer bulb is located remote from the multifunctional reservoir and is in fluid communication with the other components of the fluid transfer system with flexible tubing. The anterior wall surrounds the deflate actuator bulb and cooperates with an aperture at the apex of the deflate actuator bulb, covering the aperture to serve as an aperture valve. When the patient deforms the deflate actuator bulb the anterior wall contacts the aperture to close the aperture valve. The deflate bulb actuator is volitionally deformed causing fluid to be directed as pilot pressure to the end of a spool that is ported to deflate the at least one inflatable penile cylinder, thereby shifting the spool within the spool valve assembly actuating the deflate mechanism. The aperture valve opens in the absence of external force on the deflate actuator bulb so that the recovery of the deflate actuator bulb urges fluid to refill the deflate actuator bulb through the aperture valve. The aperture valve remains open to vent fluid from the spool valve assembly, allowing the spool to shift upon activation of the inflate mode.

In the embodiment described above, round, oval, triangular, or trapezoidal shapes with substantially parallel anterior and posterior surfaces may be adapted to achieve a 50-150 cubic centimeter volume and anatomical conformity.

The present disclosure teaches a three-piece IPP device comprising a fluid reservoir, at least one inflatable penile cylinder, a fluid transfer pump, and flexible tubing connecting the components. The fluid transfer pump comprises a fluid transfer system having an unbiased inlet valve assembly in fluid communication with the fluid reservoir, a fluid transfer bulb in fluid communication with the unbiased inlet valve assembly, an unbiased exhaust valve assembly in fluid communication with the fluid transfer bulb and at least one inflatable penile cylinder. The fluid transfer system may include a spool valve assembly in fluid communication with the fluid transfer bulb and the at least one inflatable penile cylinder. The deflate mechanism may include a spool valve assembly in fluid communication with the at least one inflatable penile cylinder and the fluid reservoir and an unbiased return valve assembly in fluid communication with the at least one inflatable penile cylinder and fluid reservoir and a deflate actuator to mechanically shift the spool valve assembly. The spool valve assembly acts as a two-position directional valve that is volitionally shifted to the cylinder inflate or cylinder deflate mode using forces external to the spool valve assembly. The spool valve assembly can be shifted to the inflate mode with hydraulic pilot pressure from the fluid transfer bulb directed at the end of the spool that is ported to inflate the at least one inflatable penile cylinder. The spool valve assembly can be shifted to the deflate mode with mechanical pressure from the deflate actuator button directed at the end of the spool that is ported to deflate the at least one inflatable penile cylinder.

### Device Operation

The present disclosure teaches the incorporation of unbiased one-way check valve assemblies into inflatable penile prostheses that may be used independently or in conjunction with other valves to either direct the flow of fluid into inflatable penile cylinders to provide a penile erection or to evacuate fluid out of the inflatable penile cylinders to cause the penis to be flaccid. Examples of unbiased check valve assemblies include IPP devices incorporating a spool valve assembly having one-touch deflate activation. Unbiased check valve assemblies would also have utility in traditional IPP devices requiring a sustained deflate activation, where they would likely be used independently of other valve assemblies.

In the cylinder inflate mode, the fluid transfer bulb is deformed providing pilot pressure to shift the spool valve to the cylinder inflate mode, after which the unbiased exhaust valve within the spool opens releasing fluid into the inflatable penile cylinders. When the deformed fluid transfer bulb is released, it recovers to its original shape. Simultaneously with recovery of the fluid transfer bulb, the unbiased inlet valve opens to admit fluid from the reservoir to fill the fluid transfer bulb and the unbiased exhaust valve within the spool closes to prevent return of fluid from the cylinders to the fluid transfer bulb. The fluid transfer bulb is repeatedly deformed and released until the inflatable penile cylinders are substantially filled and have sufficient pressure to provide an erection of the penis.

The device is placed in the cylinder deflate mode by a volitional activation of the cylinder deflate actuator. The cylinder deflate actuator, either a mechanical or pilot pressure type, is used to shift the spool valve to the cylinder deflate position. In the deflate mode fluid flows from the inflated penile cylinders through the unbiased return valve and spool valve to the fluid reservoir until the penis is flaccid. The spool valve has a fluid pathway to route the fluid from the inflatable penile cylinders to the fluid reservoir during cylinder deflation. The spool also has a fluid pathway used to route the fluid from the cylinder port in the housing to the reservoir port in the housing during device deflation. An unbiased return valve located within the spool fluid pathway opens to allow fluid to return to the reservoir and closes to prevent fluid backflow to the inflatable penile cylinders.

Various modifications and additions can be made to the exemplary embodiments discussed without departing from the scope of the present invention. While the embodiments described above refer to particular features, the scope of this disclosure also includes embodiments having different combinations of features and embodiments that do not include all of the described features. Accordingly, the scope of the present invention is intended to embrace all such alternatives, modifications, and variations as fall within the scope of the claims, together with all equivalents thereof.

What is claimed is:

1. An inflatable penile prosthesis (IPP) comprising:
a fluid reservoir for storing fluid;
at least one inflatable penile cylinder;
a fluid transfer system in fluid communication with the fluid reservoir and with the at least one inflatable penile cylinder to inflate the at least one inflatable penile cylinder with fluid from the fluid reservoir, the fluid transfer system comprising
an unbiased inlet valve assembly in fluid communication with said fluid reservoir;
a fluid transfer bulb in fluid communication with said unbiased inlet valve assembly; and
an unbiased exhaust valve assembly in fluid communication with said fluid transfer bulb and said least one inflatable penile cylinder; and
a deflate mechanism in fluid communication with said fluid reservoir and said at least one inflatable penile cylinder to return fluid from said at least one inflatable penile cylinder to said fluid reservoir, said unbiased inlet valve assembly and said unbiased exhaust valve
assembly incorporating a valve component having a specific gravity substantially similar to the specific gravity of the fluid used in said IPP.

2. The IPP of claim 1, wherein the deflate mechanism includes an unbiased return valve assembly having a valve component having a specific gravity substantially similar to the specific gravity of the fluid used in said IPP.

3. The IPP of claim 1, further comprising a spool valve assembly with a spool having an unbiased return valve assembly comprising:
a return valve chamber;
a return valve component having a specific gravity substantially similar to the specific gravity of the fluid used in said IPP; and
a return valve seat.

4. The IPP of claim 1, further comprising a spool valve assembly with a spool having an unbiased exhaust valve assembly comprising:
an exhaust valve chamber;
an exhaust valve component having a specific gravity substantially similar to the specific gravity of the fluid used said IPP; and
an exhaust valve seat.

5. The IPP of claim 1, wherein the deflate mechanism comprises a one-touch deflate mechanism.

6. The IPP of claim 1, wherein the deflate mechanism comprises a sustained deflate mechanism.

7. An inflatable penile prosthesis (IPP) comprising:
a fluid reservoir for storing fluid;
at least one inflatable penile cylinder; and
a fluid transfer system in fluid communication with the fluid reservoir and with the at least one inflatable penile cylinder to inflate the at least one inflatable penile cylinder with fluid from the fluid reservoir, the fluid transfer system comprising at least one unbiased one-way check valve assembly having a valve component with a specific gravity substantially similar to the specific gravity of the fluid used in said IPP.

8. The IPP of claim 7, further comprising a fluid transfer bulb in fluid communication with the at least one unbiased one-way check valve assembly.

9. The IPP of claim 7, further comprising a deflate mechanism in fluid communication with said fluid reservoir and said at least one inflatable penile cylinder to return fluid from said at least one inflatable penile cylinder to said fluid reservoir.

10. The IPP of claim 9, wherein the deflate mechanism includes an unbiased return valve assembly having a valve component having a specific gravity substantially similar to the specific gravity of the fluid used in said IPP.

11. The IPP of claim 7, further comprising a spool valve assembly with a spool having an unbiased return valve assembly comprising:
a return valve chamber;
a return valve component having a specific gravity substantially similar to the specific gravity of the fluid used in said IPP; and
a return valve seat.

12. The IPP of claim 11, the spool valve assembly comprising a one-touch deflate mechanism.

13. The IPP of claim 11, the spool valve assembly comprising a sustained deflate mechanism.

14. The IPP of claim 7, further comprising a spool valve assembly with a spool having an unbiased exhaust valve assembly comprising:
an exhaust valve chamber;
an exhaust valve component having a specific gravity substantially similar to the specific gravity of the fluid used said IPP; and
an exhaust valve seat.

15. A fluid transfer system for use in an inflatable penile prosthesis (IPP), the fluid transfer system comprising an unbiased one-way check valve assembly having a valve component with a specific gravity substantially similar to a specific gravity of a fluid used in said inflatable penile prosthesis.

16. The fluid transfer system of claim 15, further comprising a fluid transfer bulb in fluid communication with the at least one unbiased one-way check valve assembly.

17. A deflate mechanism for use in an inflatable penile prosthesis (IPP), the deflate mechanism in fluid communication with a fluid reservoir of the IPP and at least one inflatable penile cylinder of the IPP to return fluid from the at least one inflatable penile cylinder to the fluid reservoir, the deflate mechanism comprising a spool valve assembly and an unbiased return valve assembly having a valve component having a specific gravity substantially similar to the specific gravity of the fluid used in said IPP.

18. The deflate mechanism of claim 17, the spool valve assembly comprising a one-touch deflate mechanism.

19. The deflate mechanism of claim 17, the spool valve assembly comprising a sustained deflate mechanism.
